(19) United States
(12) Patent Application Publication
(10) Pub. No.: US 2010/0203058 A1
(43) Pub. Date: Aug. 12, 2010

(54) DIAGNOSTICS AND THERAPEUTICS BASED ON CIRCULATING PROGENITOR CELLS

(75) Inventors: David A. Ingram, Indianapolis, IN (US); Myka L. Estes, Indianapolis, IN (US); Daniel L. Prater, Indianapolis, IN (US); Laura E. Mead, Indianapolis, IN (US)

Correspondence Address: BARNES & THORNBURG LLP, 11 SOUTH MERIDIAN, INDIANAPOLIS, IN, 46204, US

(73) Assignee: Indiana University Research and Technology Corporation, Indianapolis, IN (US)

(21) Appl. No.: 12/704,275

(22) Filed: Feb. 11, 2010

Related U.S. Application Data
(60) Provisional application No. 61/151,537, filed on Feb. 11, 2009.
(60) Provisional application No. 61/222,162, filed on Jul. 1, 2009.

(51) Int. Cl. A61K 39/395; C12Q 1/02

(52) U.S. Cl. 424/141.1; 435/29

(57) ABSTRACT

Methods and compositions for detection, diagnosis, and therapeutics of arterial diseases based on pro-angiogenic and non-angiogenic circulating hematopoietic stem and progenitor cells (CHSPCs) and circulation endothelial colony forming cells (ECFCs) are described.

### CROSS-REFERENCE TO RELATED APPLICATIONS

This application claims priority to U.S. Provisional Application Ser. No. 61/151,537 filed Feb. 11, 2009 and U.S. Provisional Application Ser. No. 61/222,162 filed Jul. 1, 2009, the contents of both the applications are incorporated herein in their entireties.

### STATEMENT REGARDING FEDERALLY SPONSORED RESEARCH

Part of the work during the development of this invention was made with government support from the National Institutes of Health under grant number NIH P50 NS052606 and the Department of Defense under grant number NF073122. The U.S. Government has certain rights in the invention.

### BACKGROUND

The culture of a subset of blood mononuclear cells (MNCs) that displayed some phenotypic and functional characteristics of endothelial cells, led to the initial description of purported endothelial progenitor cells (EPCs). EPCs are thought to circulate in human peripheral blood (PB), home to sites of new blood vessel formation, and facilitate either arteriogenesis or angiogenesis by direct integration into the emerging endothelium or paracrine stimulation of existing vessel wall derived cells. EPCs, or distinct cell populations defined as circulating progenitor (CPCs), endothelial precursor (CEPs), or mature endothelial cells (CECs), have been utilized as biomarkers of human vascular disease. Using various culture methods, changes in EPC, CPC, CEP, or CEC concentration are correlated to a variety of human pathologies, including coronary artery disease, diabetes and cancer. Often, these culture techniques are laborious, expensive, and impractical for mainstream use as a diagnostic tool. While diagnostic and therapeutic decisions are being implemented based on the detection of CPCs, CEPs, and CECs in PB by traditional flow cytometry approaches, a consensus definition of these cells by analysis of surface expression of specific antigens (CD34, CD45, CD31 and AC133) is not established. As a result, various heterogeneous cell populations continue to be classified as EPCs, CPCs, CEPs, or CECs, leading to considerable confusion in both the identity of these cells and established analytical methods to assay for them in human PB.

As individual cells become increasingly analyzed by additional parameters corresponding to additional fluorescence values, the advent of polychromatic flow cytometry (PFC) emerged to implement the rigorous controls and standards necessary to discriminate multi-parametric data.

Besides occurring in frequencies on the cusp of reproducible detection (0.001-0.1%) cells of endothelial and hematopoietic origin held to engage in angiogenesis and maintain vascular health are believed to display a combination of antigens with low, dull, or a continuum of expression, and therefore analysis of these cells requires the methods of PFC. Phenotypic ambiguity calcifies a fundamental controversy within the study of endothelial progenitor cell (EPC) biology: discovering the origin and function of these purported circulating populations and debate often centers around CD45 expression in these populations, used as a measure of hematopoietic origin. The distinction between a CD34+ progenitor cell that expressed CD45 versus one that was CD34⁺ and CD45 negative was necessary for the discrimination of myeloid cells that mimicked endothelial morphology in culture (CFU-ECs) versus bona fide endothelial cells found in blood and form vessels following in vitro expansion (ECFCs). For putative EPCs, it is not clear whether CD45⁻ means CD45^{dim}. However, these debates exist because traditional flow cytometry employed to analyze these cell populations has lacked the controls necessary given the unique obstacles posed by rare event and low antigen expression profiles. Therefore, there exists a need to systematically enumerate the various subsets of hematopoietic and endothelial lineage cells and establish correlation for various disease states and develop targeted therapeutics.

### SUMMARY

A method of diagnosing cancer or peripheral vascular disease (PVD) in a subject, the method includes determining the ratio of pro-angiogenic to non-angiogenic circulating hematopoietic stem and progenitor cells (CHSPC) and diagnosing that the subject has cancer if the ratio is higher or that the subject has PVD if the ratio is lower as compared to a reference value.

In an embodiment, the ratio of pro-angiogenic and non-angiogenic circulating hematopoietic stem and progenitor cells (CHSPC) is determined by polychromatic flow cytometry (PFC). In an embodiment, the pro-angiogenic CHSPC are homogenously AC133⁺ and the non-angiogenic CHSPC are homogenously AC133⁻. In an embodiment, the pro-angiogenic CHSPC are substantially homogenous for CD45^{dim}CD34⁺CD31⁺AC133⁺CD14⁻LIVE/DEAD⁻CD41a and the non-angiogenic CHSPC are substantially homogenous for CD45^{dim}CD34⁺CD31⁺AC133⁻CD14⁻LIVE/DEAD⁻CD41a⁻.

In an embodiment, the reference value is the ratio of pro-angiogenic to non-angiogenic CHSPC of a normal, healthy sample that is substantially free of cancer and PVD. In an embodiment, the ratio of pro-angiogenic to non-angiogenic CHSPC is about 1.5 to about 3.6 for cancer and about 0.14 to about 1.52 for PVD.

In an embodiment, the pro-angiogenic CHSPC express a preponderance of myeloid markers selected from the group consisting of CD11b, CD13, and CD33 and the non-angiogenic CPCs express a preponderance of lymphoid markers selected from the group consisting of CD3, CD4, CD7, CD10, and CD56.

A method of diagnosing arterial disease, the method includes identifying microvesicles that are substantially homogenous for CD31^{bright}CD34⁺CD45⁻AC133⁻ in a sample comprising mononuclear cells, wherein the microvesicles are not endothelial cells.

In an embodiment, the identification of microvesicles is by polychromatic flow cytometry (PFC). In an embodiment, a substantial portion of the microvesicles is about 1-2 μm in diameter and are non-nucleated or anuclear. In an embodiment, the identified microvesicle population is substantially free of cells selected from a group that includes myeloid progenitors, monocytes and macrophages.

In an embodiment, the arterial disease that is diagnosed herein is cardiovascular disease.

In an embodiment, the microvesicles are selected from a group that includes endothelial microvesicles that are DAPI⁻CD45⁻CD42b⁻CD31⁺LIVE/DEAD⁻, lymphoid microvesicles that are DAPI⁻CD45⁺CD42b⁺CD31⁻LIVE/DEAD⁻, and platelet microvesicles that are DAPI⁻CD45⁻CD42b⁺CD31⁻LIVE/DEAD⁻.

A method of enumerating circulating endothelial colony forming cells (ECFCs) in a blood sample, the method includes identifying ECFCs that are homogenously CD34^{bright}CD45⁻ by polychromatic flow cytometry.

In an embodiment, the ECFCs are enumerated by bi-exponential scaling. In an embodiment the ECFCs form blood vessel in vivo through neoangiogenesis or neo vasculogenesis.

A method of reducing tumor growth (angiogenesis), the method includes decreasing the number of pro-angiogenic circulating hematopoietic stem and progenitor cells (CHSPC) in a subject suffering from or suspected of having cancer. In an embodiment, the cancer metastasis is reduced.

In an embodiment, the pro-angiogenic circulating progenitor cells (CPC) is reduced by an anti-cancer agent. In an embodiment, the anti-cancer agent is an angiogenesis inhibitor.

A method of monitoring efficacy of anti-cancer treatment, the method comprising enumerating the ratio of pro-angiogenic to non-angiogenic circulating hematopoietic stem and progenitor cells (CHSPC) in a subject undergoing anti-cancer treatment. In an embodiment, the anti-cancer treatment is selected from the group consisting of chemotherapy, antibody therapy, and radiotherapy.

### BRIEF DESCRIPTION OF THE DRAWINGS

FIG. 1 shows the characterization of CD31^{bright}D34⁺CD45⁻AC133⁻ cells. Representative PFC analysis of a CPT preparation of PB MNCs co-stained with the six-antibody/viability marker panel and the nuclear dye, DAPI (a-c). When CD14⁻glyA⁻ViViD⁻ CD31^{bright}CD34⁺CD45⁻AC133⁻ events are back-gated onto a FSC/SSC plot (blue events in a), they can be seen distributed within and below the traditional MNC threshold gate (red gate in a). (b) CD14⁻glyA⁻ViViD⁻CD31^{bright}CD34⁺CD45⁻AC133⁻ events (solid blue in b) stain negatively for the nuclear dye DAPI. Lymphocytes are overlaid in open black to show DAPI⁺ staining. (c) CD14⁻glyA⁻ViViD⁻CD31^{bright}CD34⁺CD45⁻AC133⁻ events (solid blue in c) are in the range of 1-5 μm in size. 1 (red), 3 (green), 5 (purple) and 10 (orange) μm bead standards are overlaid for reference. (d-e) Representative TEM photomicrographs of sorted CD14⁻glyA⁻ViViD⁻CD31^{bright}CD34⁺CD45⁻AC133⁻ events. Scale bars represent 500 nm. (f) CPT preparations of PB MNCs, further enriched for microvesicles (as described herein), co-stained with the six-antibody/viability marker panel and CD41a. CD14⁻glyA⁻ViViD⁻CD31^{bright}CD34⁺CD45⁻AC133⁻ events are assessed for CD41a expression. CD41a⁺ events are platelet-derived MPs (PMPs) and CD41a⁻ events are endothelial-derived MPs (EMPs). Similar results were seen in 3 independent experiments using cells from different donors.

FIG. 2 shows identification and isolation of circulating ECFCs by PFC and IMS. Representative PFC analysis of (a) adult PB MNCs and (b) CB MNCs. 500,000 events were gated for viable glyA⁻CD14⁻ cells then assessed for CD34 and CD45 expression, as shown. Putative CD34⁺CD45⁻ ECFCs are gated in green. Similar results were seen in 4 other adult and 4 other CB samples from different donors. (c-e) Immunophenotyping of purported circulating CB ECFCs for endothelial cell surface antigen expression. Cells within the green gate in (b) were sub-gated and analyzed for expression of (c) CD31, (d) CD146, and (e) CD105. Specific antigen expression is shown in filled green and unstained negative controls are shown in open black. (f-g) PFC analysis of CB MNCs (f) prior to and (g) following isolation of CD146⁺CD45⁻ cells via IMS. (h) Representative photomicrograph of an ECFC colony derived from the CD146⁺CD45⁻ fraction of CB MNCs 6 days after culture in endothelial-specific media. Scale bar represents 200 μm. (i) Low (×1,900) and (j) high (×13,000) magnification EM of sorted CB ViViD⁻CD14⁻glyA⁻CD31^{bright}CD34⁺CD45⁻AC133⁻ cells, which demonstrates the endothelial morphology of purported CB ECFCs. Scale bars represent 10 μm and 500 nm respectively. Similar results were seen in 4 other CB samples from different donors.

FIG. 3 shows identification of nonviable CECs by PFC analysis. Representative analysis of PB MNCs from breast cancer patients undergoing neoadjuvant therapy with sunitinib malate and paclitaxel stained with 7 reagents: CD34, CD45, CD31, CD146, CD105, CD14 and the viability marker ViViD. CD14⁺ events are first excluded as described herein and the remaining CD14⁻ events sub-gated onto a bi-variant plot (a) and assessed for ViViD⁻ expression. 15% of the total events are nonviable ViViD⁺ and are included with ViViD⁻ events in the bi-variant antigen plot (b) for identification of CD34^{dim}CD45⁻ cells (purple gate) and CD34^{bright}CD45⁻ ECFCs (green gate, described in FIGS. 2a-3). Cells within the purple gate in (b) are sub-gated and analyzed for expression of (c) ViViD, (d) CD31, (e) CD146 and (f) CD105. Specific antigen expression is shown in filled purple and unstained negative controls are shown in open black.

FIG. 4 demonstrates the frequency analysis of CD31⁺CD34^{bright}CD45^{dim}AC133⁺ cells. Two strategies for frequency analysis of CD31⁺CD34^{bright}CD45^{dim}AC133⁺ cells from a CPT MNC preparation of PB stained with the six-antibody/viability panel are shown. In the first strategy (a-d), manually compensated data collected on a digital flow cytometer are visualized in plots with logarithmic scaling. MNCs (red gate in a) are identified on a FSC/SSC plot and sub-gated onto a bi-variant antigen plot to identify CD34^{bright}AC133⁺ cells (dark blue gate in b). CD34^{bright}AC133⁺ MNCs are further sub-gated to identify the CD45^{dim} sub-population (light blue gate in c). CD31 expression on the resulting CD34^{bright}CD45^{dim}AC133⁺ MNCs is confirmed on a CD31 histogram (d). In the first strategy (a-d), gate boundaries are set using Boolean gating and negative isotype controls. In the second strategy (e-i), uncompensated data is collected on a digital flow cytometer, compensated after acquisition by using software, and visualized in plots with bi-exponential scaling. First, CD14⁻ cells (orange gate in e) are identified. All CD14⁻ cells are then assessed for viability and glyA expression (f). CD14⁻glyA⁻ViViD⁻ cells (pink gate in f) are sub-gated onto a bi-variant antigen plot to identify CD14⁻glyA⁻ViViD⁻CD34^{bright}AC133⁺ cells (dark blue gate in g). Viable CD14⁻glyA⁻CD34^{bright}AC133⁺ cells are further sub-gated to identify the CD45^{dim} sub-population (light blue gate in h). CD31 expression on the resulting viable CD14⁻glyA⁻CD34^{bright}CD45^{dim}AC133⁺ cells is confirmed on a CD31 histogram (i). In the second strategy (e-i), FMO gating controls (see Supplemental FIG. 8) are used to set gate boundaries.

FIG. 5 shows the characterization of CD31⁺CD34^{bright}CD45^{dim}AC133⁺ cells. (a) Semi-quantitative RT-PCR analysis of CD34, CD45, CD31 and AC133 expression in PB MNCs (▪) and sorted CD31⁺CD34^{bright}CD45^{dim}AC133⁺ cells (□). (b) Representative photomicrographs of cytospin from FACS CD31⁺CD34^{bright}CD45^{dim}AC133⁺ cells stained with Wright-Giemsa. Original magnification, 100×. (c) Representative photomicrographs of CFU-GEMM colonies derived from culture of sorted PB CD31⁺CD34^{bright}CD45^{dim}AC133⁺ cells plated in CFU-GEMM colony assays. Scale bar represents 200 μm. For all characterization assays, similar results were seen in 3 independent experiments using cells from different donors.

FIG. 6 demonstrates that CPC heterogeneity corresponds with disparate angiogenic potential and lineage markers. (a) Intravenous injection of purified CPCs results in significant increases in melanoma xenograft growth. NOD/SCID mice bearing human melanoma xenografts were intravenously injected with CD31⁺CD34^{bright}CD45^{dim}AC133⁺CPCs, CD31⁺CD34^{bright}CD45^{dim}AC133³¹ CPCs, bulk CD34⁺ cells or PBS control (n=8 per cohort) and tumor growth monitored over time in each cohort. The fold increase in tumor growth was determined by comparing tumor volume over time to base line tumor volume. *p<0.001, CD31⁺CD34^{bright}CD45^{dim}AC133⁺CPC versus CD31⁺CD34^{bright}CD45^{dim}AC133⁻CPC, CD34 or PBS control. (b) At the end of the study, tumors from each cohort of mice were harvested and weighed. *p<0.001, CD31⁺CD34^{bright}CD45^{dim}AC133⁺CPC versus bulk CD34⁺ cells or PBS control. (c) CD31⁺CD34^{bright}CD45^{dim}AC133⁺CPCs (▪) and CD31⁺CD34^{bright}CD45^{dim}AC133⁻CPCs (□) were assessed for percent expression of lymphoid (CD3, CD4, CD7, CD8, CD10, CD19 and CD45RA) and myeloid lineage markers (CD11b, CD13, CD33 and CD71).

FIG. 7 demonstrates the ratio of circulating progenitor subsets denotes disease state in peripheral vascular disease and breast cancer patients treated with sunitinib malate. Representative PFC analysis of PB MNC from healthy controls (a,d), peripheral vascular disease (PVD) patients (b) and breast cancer patients at baseline (e) and following treatment with sunitinib monotherapy (post-SU) (f) stained with the six-antibody/viability marker panel and assessed for CD34^{bright}AC133⁺ pro-angiogenic CPCs (dark blue gate) and CD34^{bright}AC133⁻ non-angiogenic CPCs (dark red gate). FMO gating controls (FIG. 15) determine gate boundaries. (c) The ratio of pro-angiogenic CPCs to non-angiogenic CPCs of PVD patients shows a significant decrease when compared to age and gender matched controls (n=9, p=0.0001). (g) The ratio of pro-angiogenic CPCs to non-angiogenic CPCs shows a significant elevation at baseline in breast cancer patients as compared to age and gender matched controls (n=9, p<0.01). Following treatment with sunitinib (post-SU), the ratio of pro-angiogenic CPCs to non-angiogenic CPCs decrease significantly as compared to both healthy controls (n=9, p<0.05) and baseline (n=9, p<0.0001).

FIG. 8 is an illustration of contour plots that allow visualization of events with minimal fluorescence. PFC analysis of a CPT MNC preparation of PB stained with antibodies directed against CD34, CD45, and CD31. Dot plot displays with logarithmic scaling (a, c) do not provide accurate visualization of events with minimal fluorescence. Contour plots of the same data (b, d) reveal many events with low or no fluorescence “piled” along the x and y axes, which were not visible in the dot plot display (indicated by red arrowheads). Similar results were seen in 9 other samples from different donors.

FIG. 9 demonstrates that the application of bi-exponential display for analysis of CD31^{bright}CD34⁺CD45⁻AC133⁻ (CECs) and CD31⁺CD34^{bright}CD45^{dim}AC133⁺ (CPCs) cells reveals a large heterogeneous event population and evidence of overcompensation. Representative PFC analysis of a CPT MNC preparation of PB stained with the six-antibody/viability marker panel (as disclosed herein). Samples were manually compensated prior to acquisition using single-color stained positive controls. Dot and contour plots shown are the analysis from a single sample. MNCs (red gate in a) are identified on a FSC/SSC plot and sub-gated onto bi-variant antigen plots (b-j). CD31^{bright}CD45⁻ (green gate in b) and CD34^{bright}CD45^{dim} (blue gate in e) cells are identified and mapped onto subsequent plots. Events displayed in logarithmic scale (b, e, h) visually appear to be properly compensated. However, conversion of this data to bi-exponential scaling (c, f, i) reveals numerous events not visible in a logarithmic display, as well as evidence of incorrect compensation (black arrowheads in c and f). Application of software-generated compensation correction yields a properly compensated display of data (d, g, j). Similar results were seen in 9 other samples from different donors.

FIG. 10 shows the effect of using single-color stained cells or compensation beads as controls to create a software-generated compensation matrix. An example of the three commonly used single-color compensation controls (a, c, e) and the resulting software compensated six-color experimental sample (b, d, f) are shown. Blue and green boxes in the top row indicate the negative and positive values, respectively, entered into the compensation matrix. Use of MNCs stained with a single antibody from the six-antibody panel (CD34 PE in a) results in under-compensation of the software compensated data (b), visualized by the asymmetric distribution of events around the yellow line. MNCs stained with CD45 PE (c) provide more distinct positive and negative populations to use in the compensation matrix, but also ultimately yield under-compensated data (d). Compensation beads incubated with individual test antibodies (CD34 PE in e) result in correct compensation as evidenced by the equivalent location of the median PE fluorescence in both the FITC negative and positive populations (f).

FIG. 11 demonstrates that the addition of fluorochromes causes increased “spreading” of data in PFC analysis. Contour plots of PB MNCs stained with APC-AF750 and APC negative isotype controls (a), CD45 APC-AF750 only (b), CD45 APC-AF750/CD34 APC/CD31 FITC (c) or CD45 APC-AF750/CD34 APC/CD31 FITC/CD14 PE-Cy5.5/glyA PacB (d). Quadrant gates (blue) based on the negative isotype staining in (a) are copied onto each subsequent plot. Panels (b-d) show CD45 APC-AF750 staining versus the APC channel, which is not stained. Symmetrical “spreading” of the CD45⁺ data points around the MFI (marked by the red vertical line in b-d) is seen as the number of fluorochromes increases. This “spreading” effect is caused by the statistical uncertainty in the measurement of fluorescence intensity introduced by compensation correction and is more apparent in multi-color stains where compensation correction between many channels is necessary. While the distribution of events appears to shift visually, the MFI in (b-d) is the same. This illustration of “spreading” is an example of the value of FMO controls for gating in PFC analysis.

FIG. 12 shows that the threshold gating of MNCs based on FSC and SSC does not remove RBC, dead cell and monocyte contamination. Representative PFC analysis of a CPT MNC preparation of PB either (a-e) stained with the six-antibody/viability marker panel (as disclosed herein), which includes glyA (RBC marker), CD14 (monocyte marker) and ViViD (cell viability marker) or (f-g) completely unstained. MNCs (red gate in a) are initially gated on a FSC/SSC plot in an attempt to exclude RBCs, dead cells and debris, and sub-gated onto a bi-variant antigen plot (b) for identification of CD31^{bright}CD45⁻ cells (green gate). CD31^{bright}CD45⁻ cells are further sub-gated to identify the CD34⁺AC133⁻ subset (orange gate in c and d). In (d), glyA⁺ViViD⁺RBCs and/or dead/apoptotic cells (purple events) and CD14⁺ monocytes (yellow events), contained within the initial MNC gate, are mapped onto the bi-variant plot and can clearly be seen distributed throughout the CD34⁺AC133⁻ gate (orange gate). Monocytes are difficult to exclude based on their scatter profile. CD14⁺ monocytes (yellow cells mapped in e) can be seen distributed throughout the MNCs when back-gated onto a FSC/SSC plot (e). In analysis of unstained samples (f-g), cells which have the scatter profile of monocytes (blue gate in f and blue cells mapped onto g), are highly auto-fluorescent and contaminate the orange gate used for frequency analysis of CD34⁺AC133⁻ cells. Lymphocytes are gated and mapped in pink for reference (f-g). Similar results were seen in 9 other samples from different donors.

FIG. 13 demonstrates that CD14 staining and a dump channel are necessary for exclusion of monocytes, RBCs and dead cells in rare cell PFC analysis. Representative PFC analysis of CPT MNC preparations of PB stained with the six-antibody/viability marker panel (a-d), glyA only (e) or ViViD only (f) or completely unstained (f-g). To exclude CD14⁺ monocytes (yellow marker in a) from analysis, all events are assessed for CD14 expression (a). CD14⁻ cells (black marker in a) are then examined for glyA expression and viability (b) to exclude RBCs and dead/apoptotic cells. CD14⁻glyA⁻ViViD⁻ cells (black events in b) are further sub-gated onto a bi-variant antigen plot (c) for identification of CD31^{bright}CD45⁻ cells (green gate). Finally, CD14⁻glyA⁻ViViD⁻CD31^{bright}CD45⁻ cells are sub-gated to another bi-variant antigen plot (d) for identification of CD34⁺AC133⁻ cells (orange gate). The resulting CD14⁻glyA⁻ViViD⁻CD31^{bright}CD45⁻CD34⁺AC133⁻ population is markedly different than the CD31^{bright}CD45⁻CD34⁺AC133⁻ population achieved via threshold gating on scatter plots (see FIG. 3a-c). Back-gating of cells singly stained for glyA (e) or ViViD (f) onto a FSC/SSC plot shows glyA⁺ RBCs (pink cells in e) and ViViD⁺ dead/apoptotic cells (blue cells in f) distributed throughout the MNC gate (red gate), therefore RBCs and dead/apoptotic cells cannot be reasonably excluded based solely on their scatter profile. Similar results were seen in 9 other samples from different donors.

FIG. 14 demonstrates that logarithmic scaling does not allow the visualization of CD45 negative populations.

FIG. 15 shows FMO gating controls for analysis of CD31⁺CD34^{bright}CD45^{dim}AC133⁺ cells. Shown are the corresponding FMO gating controls necessary for the frequency analysis of CD31⁺CD34^{bright}CD45^{dim}AC133⁺ cells in PB (as in FIG. 8e-i). Each panel shows MNCs stained with all reagents in the six-antibody/viability marker panel except for one: (a) lacks AC133 APC, (b) lacks CD45 APC-AF750, (c) lacks CD31 FITC, and (d-e) lacks CD34 PE. Black gates in (a), (b), and (d-e) delineate the positive staining threshold for APC, APC-AF750 and PE, respectively. Blue gates in (a-b) and (d-e) are the actual gates used for frequency analysis of CD31⁺CD34^{bright}D45^{dim}AC133⁺ cells when MNCs are stained with the full six-antibody/viability marker panel (as in FIG. 8g-i). The black marker in (c) delineates events that are positive for FITC staining.

FIG. 16 shows immunolectron microscopy of cultured CB ECFCs and PFC sorted CB ViViD⁻CD14⁻glyA⁻CD31^{bright}CD34⁺CD45⁻AC133⁻ cells for vWF. Representative electron micrographs of (a) cultured CB ECFCs and (b) sorted CB ViViD⁻CD14⁻glyA⁻CD31^{bright}CD34⁺CD45⁻AC133⁻ cells stained with the immunogold method using polyclonal anti-vWF antibody. Positive gold labeling (black arrows) indicates the presence of vWF. Original magnification, ×30,000. Scale bar represents 500 nm. Similar results were seen in 4 other CB samples from different donors.

### DETAILED DESCRIPTION

Methods and compositions disclosed herein identify whether human circulating pro-angiogenic cells represent a subset of the hematopoietic system and express CD45 or are hematopoietic derivatives that do not express CD45 (and are called endothelial progenitor cells). Polychromatic flow cytometry (PFC) protocols have been developed to isolate subsets of hematopoietic cells and are used to identify the circulating pool of CD34⁺CD45^{dim} cells representing functional circulating hematopoietic stem and progenitor cells (CHSPCs) that are separated on the basis of AC133 expression.

A novel polychromatic flow cytometry (PFC) protocol for enumeration of circulating human hematopoietic cells, circulating progenitor cells and endothelial colony forming cells (ECFCs) is described. These are characterized by cell surface antigen expression, colony assay, morphological analysis including EM, and in vivo function. The circulating hematopoietic cells and ECFCs identified are validated herein, or in other studies, as cells that function in neoangiogenesis and serve as biomarkers of cardiovascular disease (CVD) or tumor progression. Detection of human CECs using conventional flow cytometry and analytical methods is now clarified to identify circulating platelet and endothelial microvesicles that are devoid of endothelial cells. Circulating progenitor cell (CPC) enumeration that correlates with tumor progression risk is clarified herein to identify hematopoietic progenitor cells, myeloblasts and engrafting hematopoietic stem cells (HSCs). Data is presented that re-defines the analytical method for enumerating circulating blood cells that participate in new blood vessel formation at homeostasis and in subjects with abnormal cardiovascular health. A novel PFC protocol is disclosed herein as a unifying approach that enables investigators and practicing physicians to specifically identify which blood and endothelial cell subsets function in human cardiovascular health and disease.

In an embodiment, the AC133⁺ subset of the CHSPCs enhances the growth of tumor blood vessels in vivo in immunodeficient mice. In an embodiment, the ratio of AC133⁺ pro-angiogenic CHSPCs to AC133⁻ non-angiogenic CHSPCs provides a statistically significant correlation with the severity of the clinical state of patients with peripheral arterial disease (PAD). Methods disclosed herein, validated via in vitro and in vivo analyses, are used to interrogate the roles of human hematopoietic elements in the growth and maintenance of the vasculature.

In an embodiment, the CPC subset was identified as CD45^{dim}CD34⁺CD31⁺ and heterogeneous in AC133 expression and includes circulating hematopoietic stem and progenitor cells (CHSPCs) that engraft in NOD/SCID mice of which a subset display pro-angiogenic tumor growth promoting activity in vivo.

Confusion around the function of, EPCs, circulating endothelial progenitors (CEPs), and CPCs in vascular repair and regeneration at homeostasis or in response to injury or disease is linked to lack of consensus regarding quantitative measures to isolate each cell type using in vitro colony assays, immunomagnetic separation (IMS), or conventional flow cytometry approaches. Use of the term CPC, without functional validation of the cell types comprising this fraction has not been helpful in understanding the mechanisms of cellular action purported to emerge from these flow cytometry “events”. A new approach in defining the parameters and properties of cells involved in neoangiogenesis is required for advancements in clinical treatments.

In an embodiment, the CPC population identified using the conventional flow cytometry approach and the novel CHSPC population isolated using the PFC protocol are demonstrated herein to include hematopoietic cells at different stages of differentiation. Substantially all observed cells belong to the HSPC pool, a substantially significant proportion of which display in vitro hematopoietic CFC activity and others are capable of engrafting in immunodeficient mice. Hematopoietic cells participate in angiogenesis. Increased concentrations of CPCs correlate with risk for tumor recurrence and patient responsiveness to anti-angiogenic therapies.

Methods disclosed herein including the use of the PFC protocol have enabled to distinguish CHSPCs with pro-angiogenic function and those lacking in angiogenic supportive activity, based upon AC133 expression. AC133 is proposed as a marker for circulating EPCs and has been used in combination with CD34 and/or CD31 and/or KDR as a biomarker in patients with CVD, cancer, sepsis, or renal failure. The PFC approach reported herein, permits isolation of CHSPC subsets based upon AC133 expression and only the CD31⁺CD34^{bright}CD45^{dim}AC133⁺ CHSPC subset possesses pro-angiogenic activity in promoting angiogenesis and human melanoma tumor growth in an immunodeficient mouse explant model system. This particular CHSPC subset was enriched in cells displaying a variety of myeloid cell surface antigens in addition to displaying in vitro and in vivo HSPC functions. This subset did not display any vasculogenic ability in vivo when examined for the presence of human endothelium within the explanted human tumors within the immunodeficient mice. Thus, the pro-angiogenic CHSPC is substantially enriched in pro-angiogenic functions but lacks postnatal vasculogenic activity and permit a better understanding of mechanisms for blocking tumor angiogenesis.

In an embodiment, a variance of circulating concentrations of pro-angiogenic and non-angiogenic CHSPCs was observed in patients with PAD. A significant decrease in the ratio of pro-angiogenic to non-angiogenic CHSPCs was measured in the bloodstream of patients with PAD as compared to healthy control subjects. Differences in the gene expression and function of the CHSPCs in normal subjects and those with PAD at various stages of their disease are helpful in identifying agents to block disease initiation and progression.

The data disclosed herein support methods for identifying distinct subsets of circulating cells in human PB that promote angiogenesis. Prospective identification of these cells facilitates human clinical studies and functional biological characteristics of each defined cellular subset.

In an embodiment, the content of CPCs was assayed by PFC in the PB of adolescent type I diabetic patients as a biomarker of early vascular disease. Decreased CPCs were detected in all type I diabetic patients compared to controls, which correlated better with metrics of endothelial cell dysfunction than hemoglobin A1C levels. The specific function of each hematopoietic cell subset in tumor angiogenesis and vascular repair is being elucidated.

In an embodiment, the ability to specifically identify and enumerate the rare circulating ECFCs in human PB is reported herein. ECFCs with robust proliferative potential are identified in cord blood (CB). Further proof that the samples contain proliferative cells was obtained when using CD146 beads and IMS to isolate ECFCs from the CD45⁻ and not CD45⁺ fraction of blood samples. Thus, ECFCs, the only EPC with in vivo blood vessel forming capacity, are circulating in CB and can be detected by the PFC protocol disclosed herein. Immunomagnetic separation (IMS) is suitable for ECFC isolation if clonal assays and propagation are needed for in vitro and in vivo applications including blood vessel formation and for assaying candidate agents and drugs in vitro.

In an embodiment, in human subjects with peripheral arterial disease and adolescent type I diabetics with early vascular disease, ECFCs are detected using the PFC protocol, which indicates that ECFCs are mobilized in these diseased subjects similar to previous observations in patients with progressive coronary artery disease. CB CD146⁺ cells that give rise to ECFCs are enriched in the CD45⁻ rather than the CD45⁺ fraction. This result may have been due to the CD45 depletion studies (removing T cells) prior to the CD146 selection.

Understanding neoangiogenesis and the role of specific cell or sub-cellular elements are useful in the field of cardiovascular biology. This is now possible through the PFC analysis and results disclosed herein that unify rather than confuse the understanding of neoangiogenesis and the role of specific cell or sub-cellular elements that have been erroneously masked by the term EPC. Due to the confusion and lack of specificity surrounding the term EPC, as discussed herein ECFC is a suitable term to describe the subset of circulating and resident endothelial cells that inherently possess the capacity to form the new blood vessels and that hematopoietic cell subsets, which participate in angiogenesis or vasculogenesis, be identified by their appropriate lineage designation. This specificity permits a rational strategy for identifying which cells are defective in patients with CVD and for selecting the appropriate replacement cell therapy for tissue repair and regeneration.

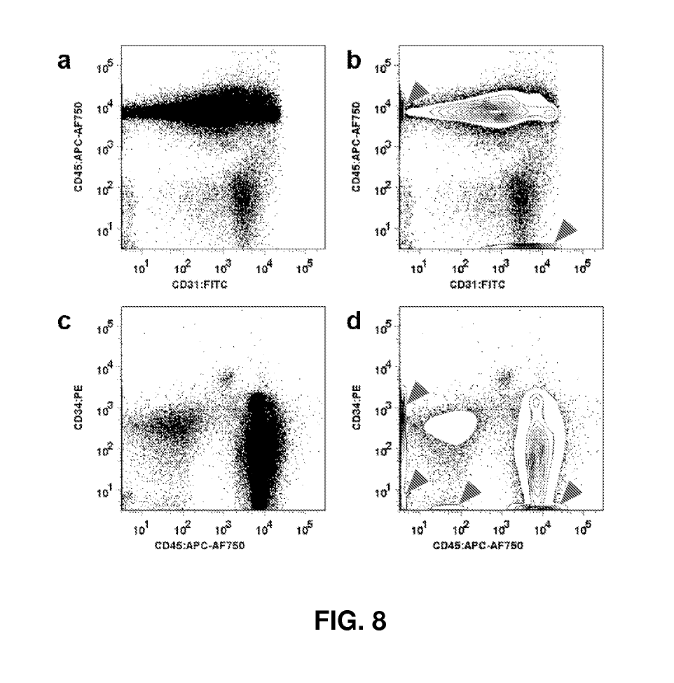

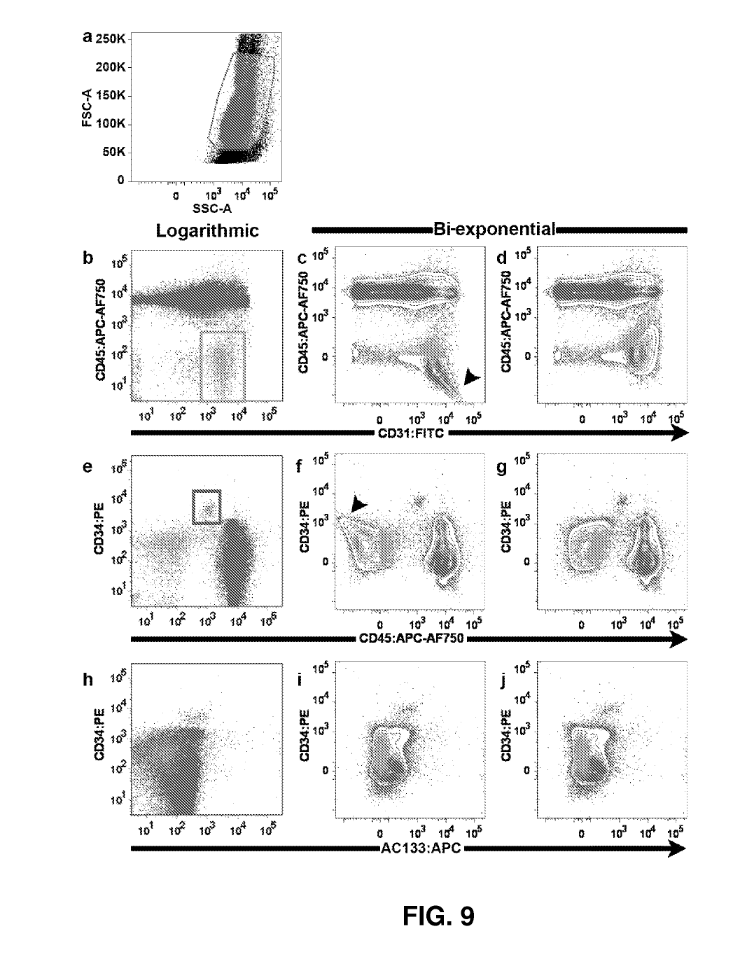

EPC and CEC traditional flow cytometry data reveal manual overcompensation, log amp conversion errors, poor resolution and sensitivity, logarithmic visualization artifacts and insufficient discrimination between cell types, background, autofluorescence and contaminating false positives that affect the sensitivity and specificity. The compensation errors may be tolerated if the population of interest displays uniform and abundant expression of the staining antigens. However, enumerating a population with low or negative expression (e.g., CD34, AC133 and CD45, respectively), user-generated errors may invalidate cellular measurements. These substantial errors may have been overlooked due to the visual distortions of conventional logarithmic dot display, masking relevant populations that fall near or below the baseline (see FIGS. 8-9).

PFC analysis generally requires the use of standard software programs to achieve optimal compensation. To calculate the compensation correction, distinct positive and negative values for each fluorochrome are entered into a compensation matrix. Single-color controls are run independently to generate these values. Generally, cells stained with the individual antibodies from a test panel are used as the single-color controls to set compensation correction, either visually or with computer software. Errors occur particularly when antibodies against dimly or lowly expressed antigens (e.g. CD34 or AC133) are used as controls because distinct positive and negative populations are not present. Additionally, cells stained for dimly expressed antigens are insufficient controls because the range of compensation correction is only valid up to the brightest events within the positive population of the single-color control. Cells that may stain brighter in the experimental sample may not be reliably compensated.

In an embodiment, a compensation control is MNCs singly stained with a CD45 antibody conjugated to each of the fluorochromes corresponding to the experimental antibodies. While these single-color controls provide distinct positive and negative populations and bright positive staining, they may be inadequate controls. Compensation matrices are based on the protein to fluorochrome ratio of each individual antibody. Therefore, compensation correction calculated based on a series of CD45 stained cells may not be applicable to an experimental sample stained with a different series of antibodies.

In an embodiment, for compensation correction, compensation beads incubated with the individual antibodies used in the experimental multi-color sample may be used. Compensation beads prepared in this manner provide distinct negative and brightly positive values for the matrix, as well as account for the protein to fluorochrome ratio of each reagent used.

In an embodiment, bi-exponential or ‘logicle’ scaling enables the visualization of all events, highlights compensation errors and eliminates deceptive data spreading (See FIG. 9). Bi-exponential scaling enables determining populations with low to negative fluorescence and visually confirming proper compensation. For example, a truly CD45 negative population (e.g., involved in neovasculogenesis) may not be reliably verified using conventional flow cytometry displays. However, when applied to the analysis of putative CECs, measurements detecting a seemingly homogeneous CD31^{bright}CD34⁺CD45⁻AC133⁻ population includes an artificial subdivision of a larger heterogeneous event population: an artifact of logarithmic scaling and severe over-compensation (see FIG. 9). These errors preclude the conventional standards of manual compensation, isotype gating controls and quadrant analysis. These errors therefore render conventional EPC/CEC flow data collected and analyzed employing traditional flow cytometry techniques less reliable.

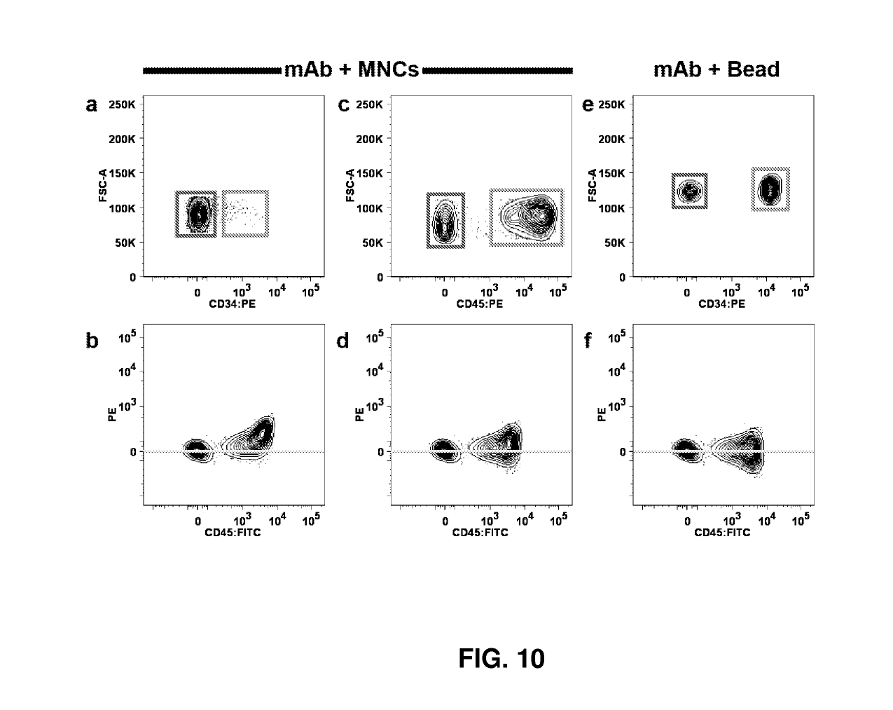

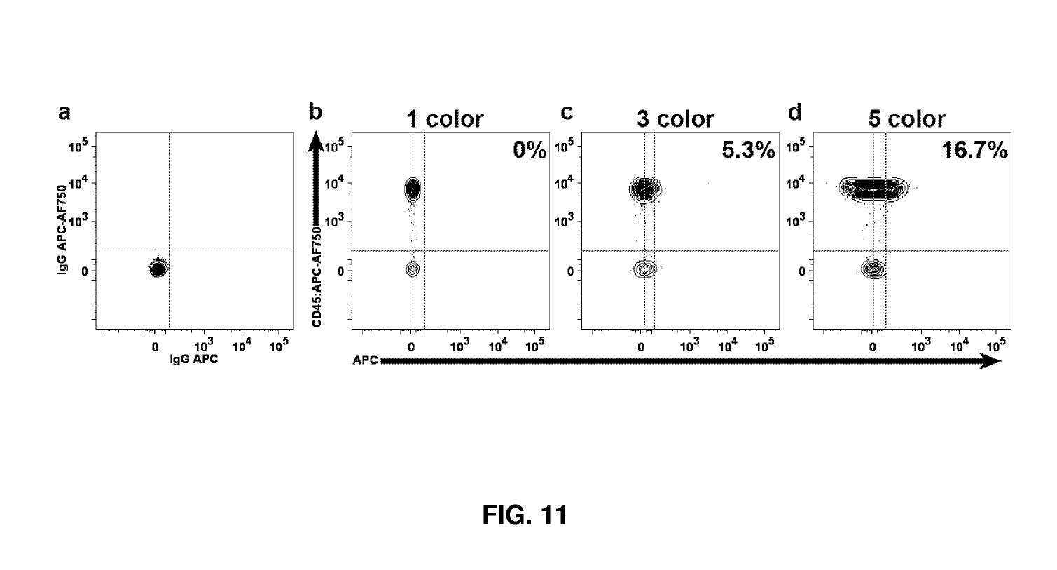

In an embodiment, identification of rare cells (e.g., ECFCs) requires post-acquisition compensation via a software-generated matrix based on compensation bead controls (see e.g., FIG. 10 and accompanying description). Following the application of correct compensation, objective gating controls are used to determine the threshold separating negative populations from dully fluorescent cells. ‘Fluorescence minus one’ (FMO) controls, generated by staining cells with all the reagents except the one for which the positive threshold is determined, are included whenever the population bounds are inexplicit (FIG. 11). The use of isotype controls is not favored clinically, especially in rare event analysis.

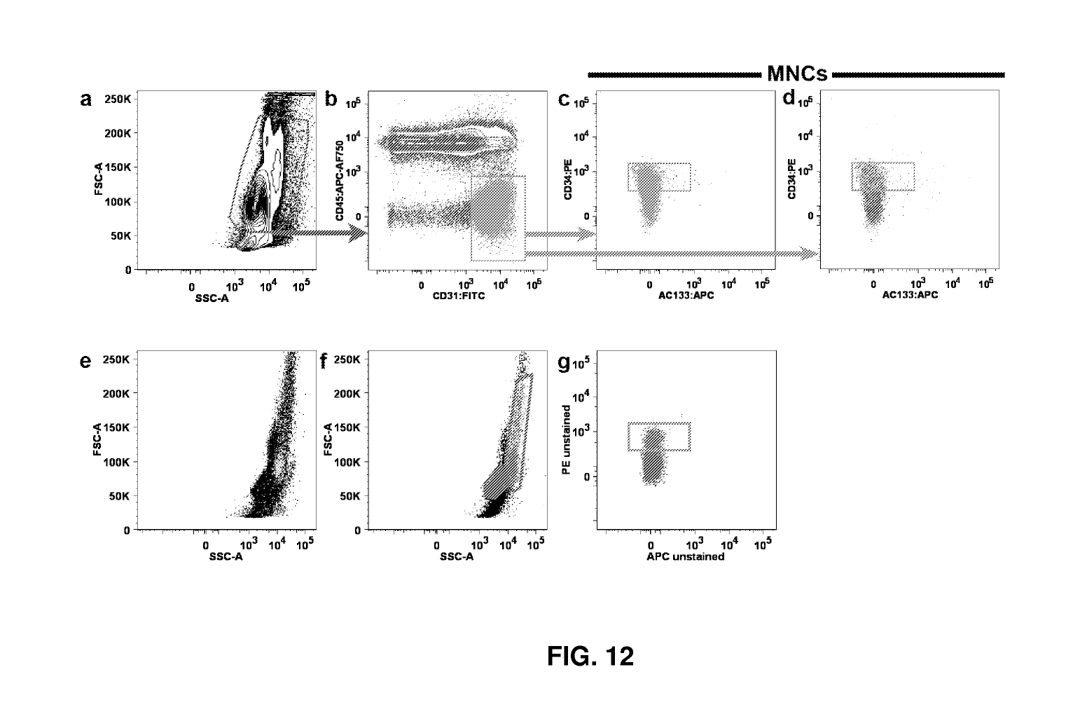

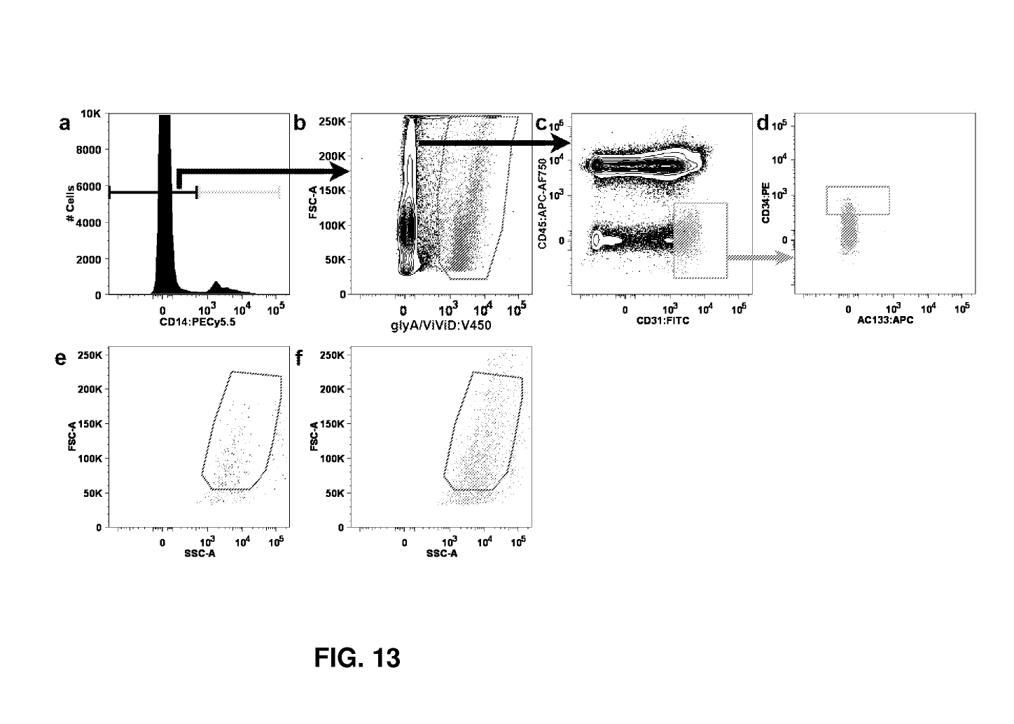

In an embodiment, to identify putative EPCs and CECs, exclusion of myeloid progenitors, monocytes and macrophages is used in rare cell flow cytometry analysis where contamination of cell populations with false positive and non-specific fluorescent events; monocytes, red blood cells (RBCs), and dead cells auto-fluoresce and non-specifically bind numerous antibodies is encountered. In an embodiment, at least 95% of these contaminants are excluded from enumeration. In an embodiment, more than about 90% or 95% or 96% or 97% or 98% or 99% of the contaminants are excluded from the rare cell analysis. Flow cytometry protocols employ either a forward scatter (FSC) threshold gate or an inclusive MNC gate as the initial analysis step may not be sufficient to eliminate confounding cellular contaminants (FIGS. 12-13). Since putative EPCs and CECs circulate at frequencies (0.001-0.1%) significantly below the fraction of common contaminants (5-10% see (FIGS. 12-13), the exclusion of these unwanted events may be necessary to ensure that reported populations are not mere artifacts. Additionally, the simultaneous analysis of populations of differing autofluorescence may mask rare populations and results in contaminating false positives (FIG. 12). Many purportedly endothelial-specific in vitro and in vivo assays show myeloid lineage contamination, undermining the knowledge and data behind EPC biology. Therefore, proper identification and exclusion of myeloid progenitors, monocytes and macrophages in any technique used in identifying putative EPCs and CECs is helpful.

The terms pro-angiogenic and non-angiogenic circulating progenitor cells as used herein relate as follows:

Pro-angiogenic: CD45^{dim}CD34⁺CD31⁺AC133⁺CD14⁻LIVE/DEAD⁻CD41a⁻ Non-angiogenic: CD45^{dim}CD34⁺CD31⁺AC133⁻CD14⁻LIVE/DEAD⁻CD41a⁻

Microvesicles as used herein relate to:

Endothelial Microvesicles: DAPI⁻CD45⁻CD42b⁻CD31⁺LIVE/DEAD⁻
Lymphoid Microvesicles: DAPI⁻CD45⁺CD42b⁻CD31⁻LIVE/DEAD⁻
Platelet Microvesicles: DAPI⁻CD45⁻CD42b⁺CD31⁻LIVE/DEAD⁻

ECFCs are CD34^{bright}CD45⁻AC133⁻CD31⁺CD146⁺.

The term substantially homogenous refers to nearly uniform expression of the tested surface markers subject to any minimal variation caused by insignificant contaminants and any culture artifacts. For example, if at least 99% or 98% or 97% or 96% or 95% of a population of the cells express a given set of the markers, the population of the cells is characterized as being substantially homogenous for those markers.

An isolated population of cells as used herein refers to a distinct substantially homogenous population of cells that express a selected group of markers including surface antigens and are functionally capable of displaying a specific attribute, such as colony formation from a single cell.

The term consisting essentially or consists essentially of refers to a select population or sub-population of cells that exhibit a particular marker profile and/or capable of displaying a specific attribute and any other cell population that does not materially alter the function of the select population of cells.

The term reference value refers to a control value of the ratio of pro-angiogenic to non-angiogenic circulating hematopoietic stem and progenitor cells (CHSPC) based on a determination of an average ratio from healthy samples that are generally free of vascular/arterial diseases and cancer.

### EXAMPLES

### Example 1

### Frequency Analysis and Characterization of CD31^{bright}CD34⁺CD45″AC133″ Cells (CECs)

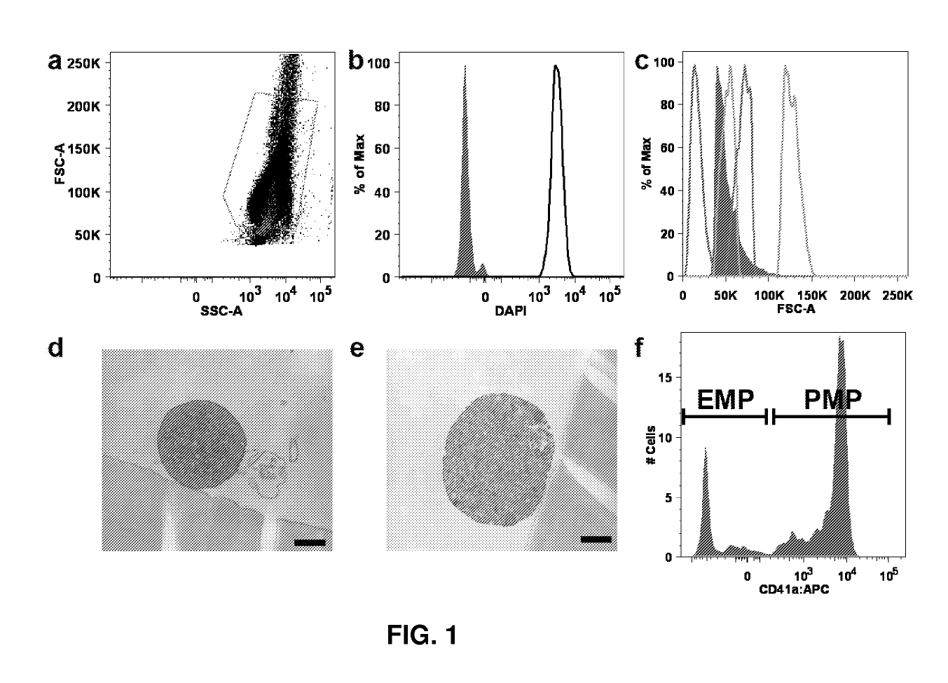

The PFC methodology outlined herein identifies some of the significant problems in determining the frequency and phenotype of CECs with use of traditional flow cytometry analysis strategies (see e.g., FIGS. 8-14). When the reported CEC population is validated with proper scaling and compensation, the observed frequency was too high to be a homogeneous population of CECs (FIG. 9). Based on this surprising observation and potential misidentification of purported CECs utilizing prior protocols, the cellular identity of CD31^{bright}CD34⁺CD45⁻AC133⁻ events was determined by PFC. Back-gating of CD31^{bright}CD34⁺CD45⁻AC133⁻ events onto the original FSC/SSC dot plot revealed their location spanning the lower margin of the MNC gate in a region that typically harbors small cells and debris and not endothelial cells (blue events in FIG. 1a). To determine the percentage of CD31^{bright}CD34⁺CD45⁻AC133⁻ events that were cells, the population was stained with a DAPI nuclear stain. Utilizing lymphocytes as a positive cellular control, none of the CD31^{bright}CD34⁺CD45⁻AC133⁻ events contained a nucleus, which confirmed that these events were in fact not endothelial cells (FIG. 1b).

Microvesicles do not contain a nucleus, are small in FSC/SSC dot plots, and are shed from endothelial cells, platelets, lymphocytes, monocytes and other leukocytes. Microvesicles are generally excluded by threshold gating in FSC, however, setting the FSC lower bound to include the entire lymphocyte population selects for microvesicle and platelet aggregates which form under conditions of activation, a process artificially exacerbated by certain anticoagulants. Microvesicles are biologically active and correlate with cardiovascular disease (CVD) risk. It was tested whether the CD31^{bright}CD34⁺CD45⁻AC133⁻ events were microvesicles. As shown in FIG. 1c, when CD31^{bright}CD34⁺CD45⁻AC133⁻ events are compared with beads of standardized size in flow cytometry analysis, a substantial majority of CD31^{bright}CD34⁺CD45⁻AC133⁻ events are 1-2 μm in diameter. Analysis of the CD31^{bright}CD34⁺CD45⁻AC133⁻ events by electron microscopy (EM) revealed the typical phenotypic morphology of microvesicles (FIG. 1d-e). Additionally, EM confirmed the formation of aggregates, which account for anuclear events that are greater than 2 μm in size and overlap intact lymphocytes in the MNC gate. Microvesicles are generally identified by flow cytometry as leukocyte-, endothelial-, or platelet-shed particles by determining the number of CD45⁺CD41a⁻ (leukocyte), CD45⁻CD31⁺CD34⁺CD41a⁻ (endothelial), or CD41a⁺CD31⁺ (platelets) events. Flow cytometry analysis of the microvesicles contained in the CD31^{bright}CD34⁺CD45⁻AC133⁻ gate revealed a heterogeneous population of endothelial and platelet microvesicles (FIG. 10. Collectively, this data generated by application of PFC demonstrates that the description of CECs is flawed and that CD31^{bright}CD34⁺CD45⁻AC133⁻ events are largely composed of endothelial- and platelet-derived microvesicles.

### Example 2

### Prospective Identification and Isolation of Circulating ECFCs by PFC

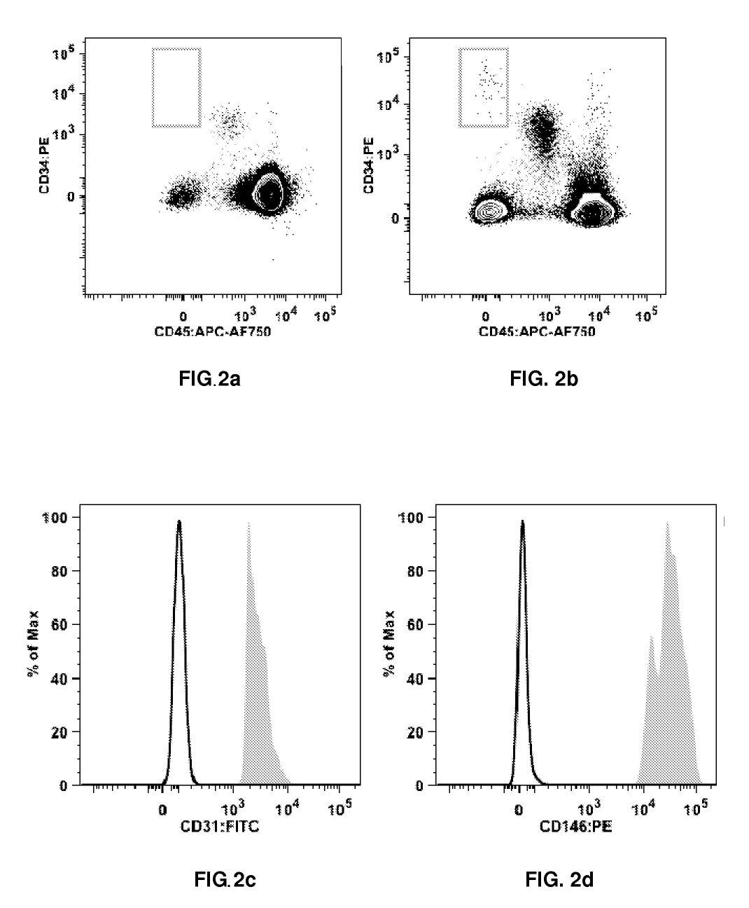

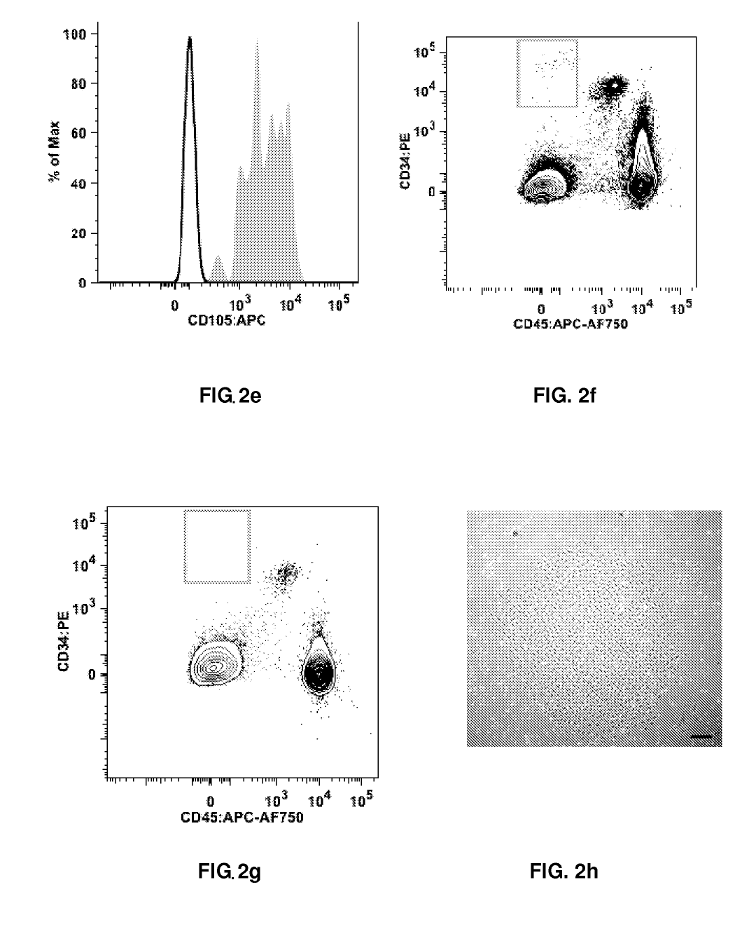

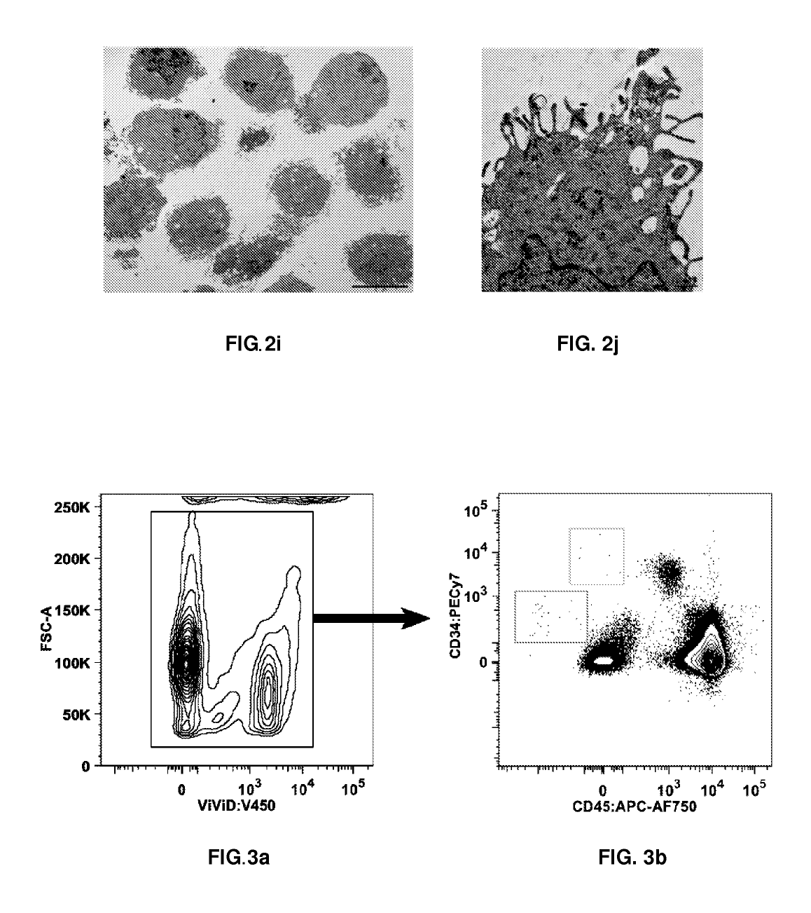

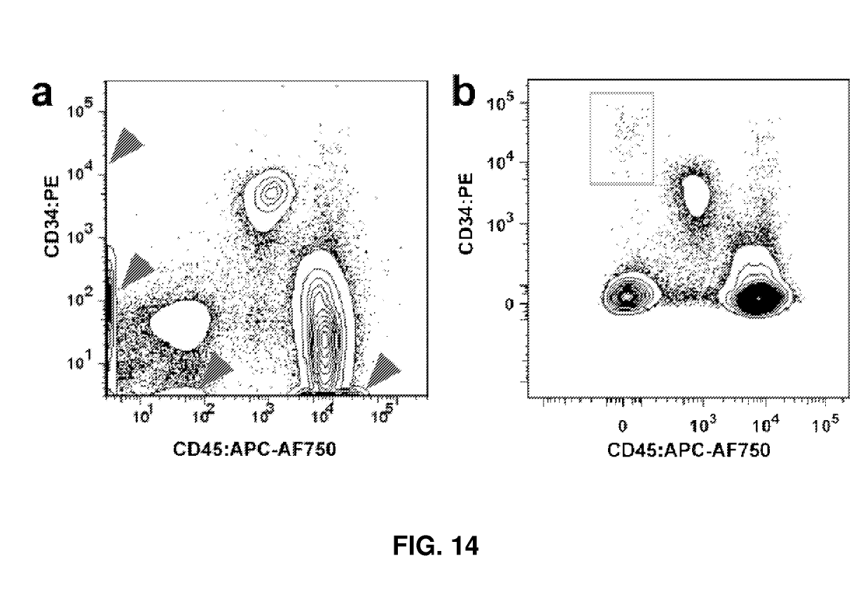

As disclosed herein, a definitive method for prospective isolation of ECFCs or CECs by flow cytometry is not currently available. ECFCs are rare circulating EPCs with clonogenic and in vivo vessel forming capacity, and CECs have limited proliferative potential. ECFCs and/or CECs were isolated by PFC in human umbilical cord blood (CB). ECFCs express CD34 and not CD45 and can be enriched by immunomagnetic separation (IMS) for these defined cells (CD34⁺CD45⁻). MNCs derived from adult PB and CB were stained with antibodies directed against CD34 and CD45 to identify CD34^{bright}CD45⁻ cells. CB MNCs harbored a small population of viable CD34^{bright}CD45⁻ cells (FIG. 2a-b). Further, CB derived CD34^{bright}CD45⁻ cells co-express the endothelial cell surface antigens CD31, CD146, and CD105 (FIG. 2c-e), that are present on circulating ECFCs and CECs. As discussed herein, in logarithmic scaling, these CD34^{bright}CD45⁻ cells are compressed against the baseline and remain hidden even with the application of proper compensation (FIG. 14). Thus, detection of this rare population in this experiment requires bi-exponential scaling and cannot be resolved using conventional techniques. To determine if this cell population would form endothelial cell colonies after prospective isolation and culture in defined endothelial cell media, CB derived CD146⁺CD45⁻ cells were isolated by a two step magnetic bead cell sorting procedure (CD45⁺ cell depletion followed by CD146⁺ enrichment of the CD45⁻ fraction). IMS was utilized so that ECFCs survive the isolation process. As shown in FIG. 2f-g, PFC analysis of CB MNCs before and after IMS demonstrated that this procedure captured the entire CD34^{bright}CD45⁻ cell population. PFC analysis of the isolated cells confirmed that they are homogenous CD146⁺CD45⁻ cells and also expressed CD34, CD31 and CD105, but not AC133.

The IMS-sorted CD146⁺CD45⁻ cells were cultured in defined endothelial and hematopoietic cell culture conditions for colony formation. CD146⁺CD45⁻ cells formed multiple distinct ECFC colonies within 3-4 days (FIG. 2h) and never yielded hematopoietic progenitor cell colonies (n=4). To further confirm that the CD34^{bright}CD45⁻ population identified by PFC prospectively identifies the putative ECFC, FACS was used to identify CD34^{bright}CD45⁻ cells and analyzed the sorted cells by EM. Substantially all of the cells were homogenously endothelial cells as determined my EM examination (FIG. 2i-j). Thus, PFC identifies CD34^{bright}CD146⁺CD31⁺CD105⁺CD45⁻ cells, which contain highly proliferative but rare ECFCs.

### Example 3

### Prospective Identification of Nonviable CECs by PFC

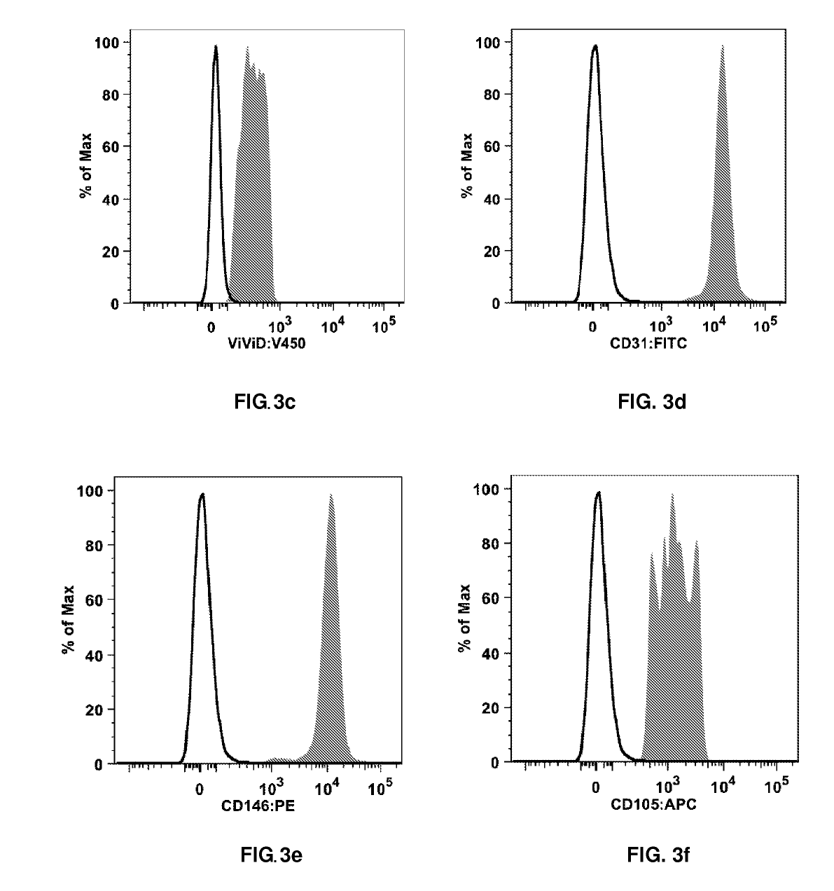

As discussed herein, false positives due to the non-specific binding of dead cells to antibody conjugates leads to considerable error in rare event analysis. However, substantial data indicates that putative CECs reflect vascular injury, expressing markers of early apoptosis or necrosis. The excluded ViViD positive fraction for evidence of this population in metastatic breast cancer patients undergoing neoadjuvant therapy with sunitinib malate and paclitaxel were studied. PB MNCs from breast cancer patients were stained with six monoclonal antibodies (CD34, CD45, CD31, CD146, CD105 and CD14) and the viability marker (ViViD) with FMO controls. Examination of CD34, CD45 bi-variant contour plots revealed an unseen population of ViViD^{dim}CD34^{dim}CD45⁻ as well as the ViViD⁻CD34^{bright}CD45⁻ population observed in the CB samples (FIG. 3a-c). The ViViD^{dim}CD34^{dim}CD45⁻ falls into the negative decades beyond that of other CD45 negative populations as a by-product of compensation and beginning autofluoresence levels in other parameters and is not an indication of degrees of negativity. Further phenotyping reveals CD31, CD105 and CD146 expression confirming mature endothelial morphology with limited or no clonogenic potential (FIG. 3d-f).

### Example 4

### Frequency Analysis and Characterization of CD31+CD34^{bright}CD45^{dim}AC133⁺ cells (CPCs)

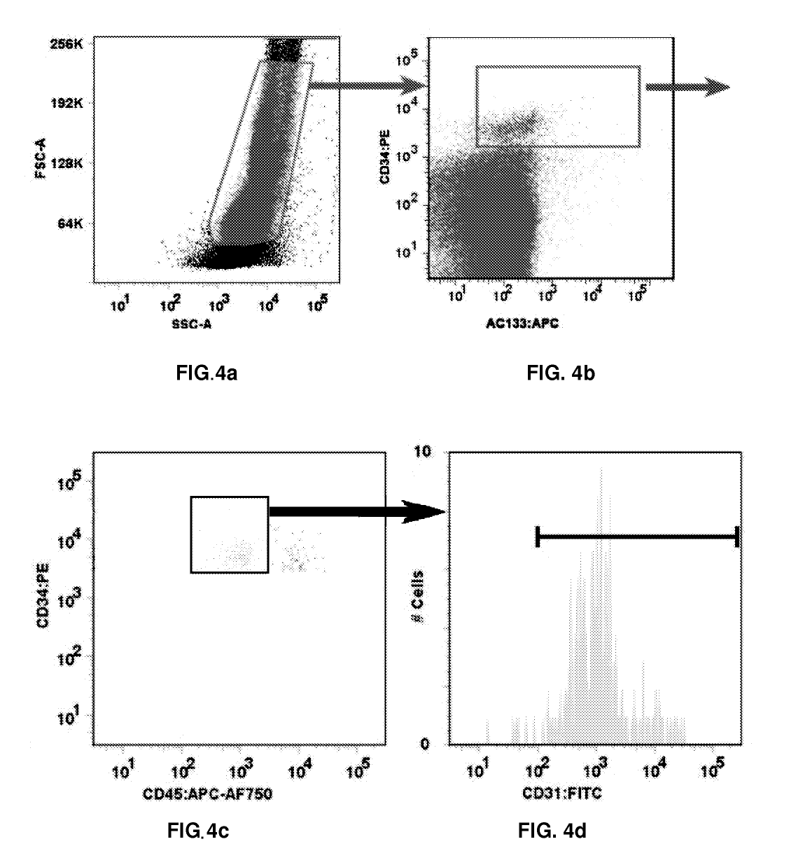

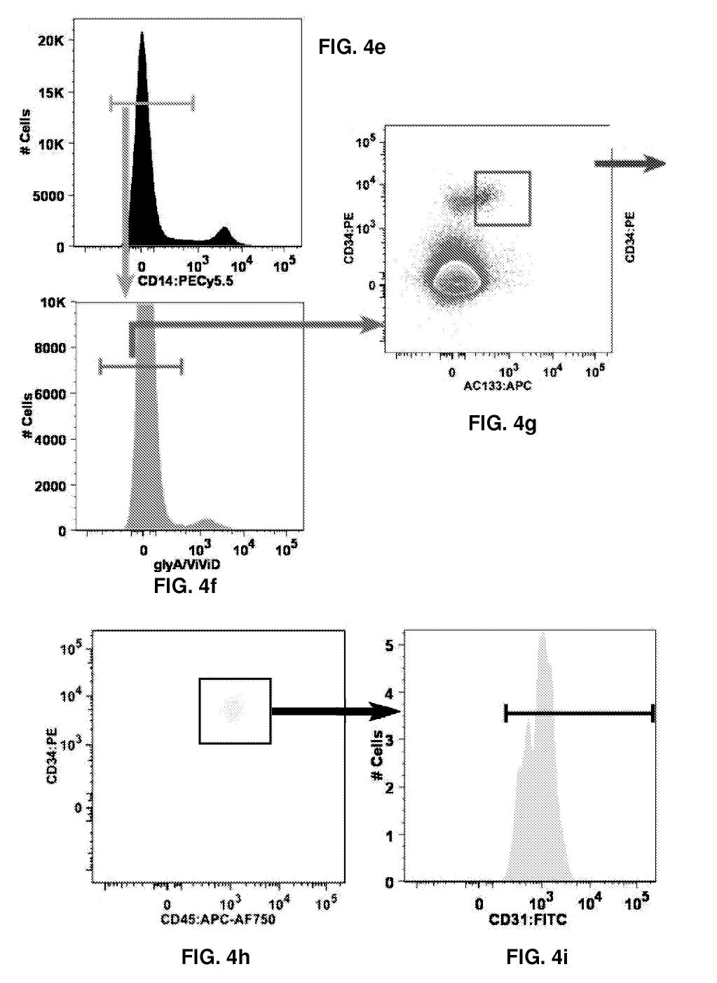

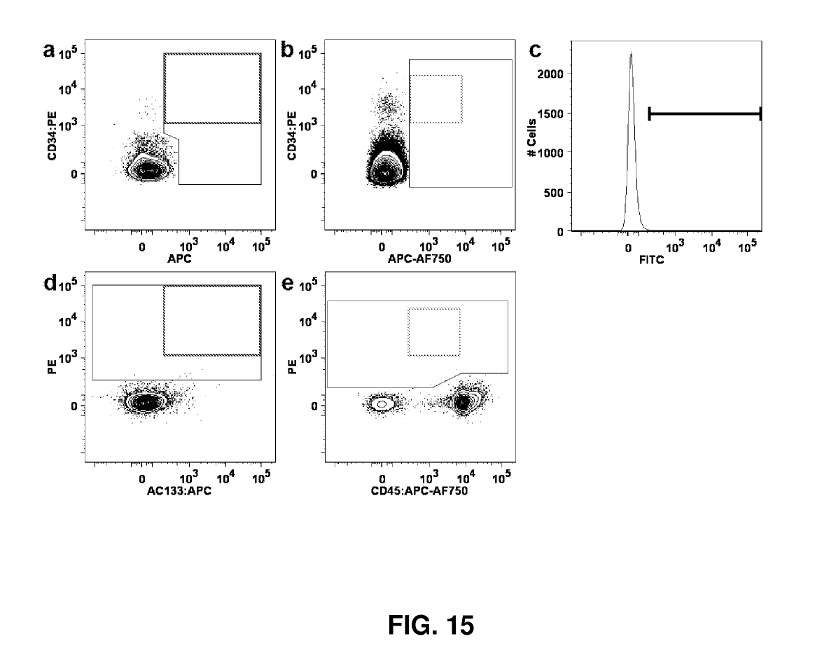

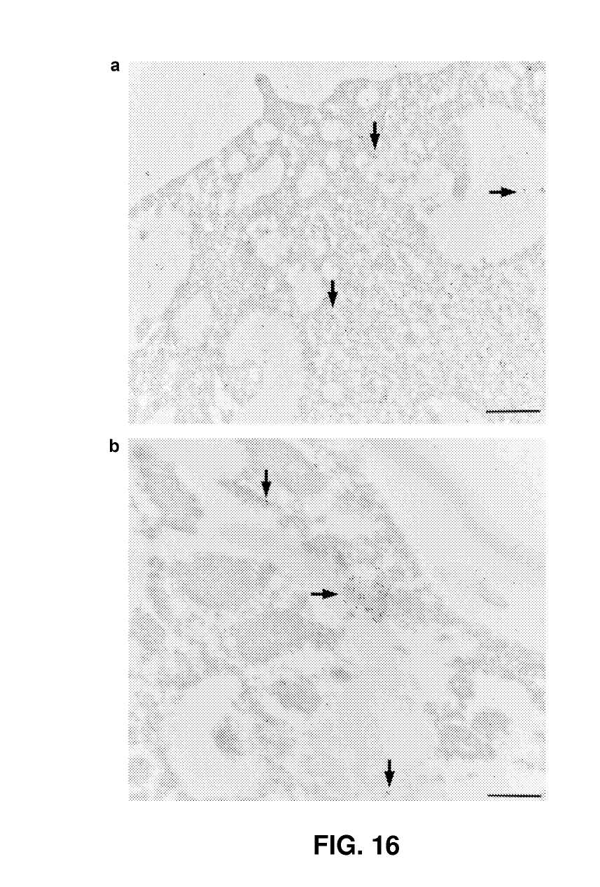

A methodological comparison for the flow cytometry enumeration of CD31⁺CD34^{bright}CD45^{dim}AC133⁺ putative CPCs was performed. PB MNC samples were isolated from 10 healthy, young adults and were stained with the six monoclonal antibodies (CD34, CD45, CD31, AC133, glyA, and CD14) and the viability marker (ViViD) with FMO controls or isotype controls. Stained samples were acquired on a digital BD LSRII flow cytometer and assessed for CD31⁺CD34^{bright}CD45^{dim}AC133⁺ (CPCs) events using two different analysis schemas as shown in FIG. 4. First, stained MNCs were analyzed in logarithmic dot plots (FIG. 4a-d) and CPC identification was determined by placement of population gates, which were based on isotype controls (FIG. 4a-d). Data were manually compensated using singly stained cell controls. In contrast, MNCs from the same donor were analyzed in bi-exponential contour plots to identify CD31⁺CD34^{bright}CD45^{dim}AC133⁺ cells after exclusion of contaminating monocytes, RBCs and dead cells (FIG. 4e-i). Prior to analysis, automated compensation was applied based on single-color bead controls (FIG. 10). To objectively identify CPCs, regional gates were applied based on the use of proper FMO gating controls (FIG. 15).

To compare the reproducibility and margin of error between the two methods, PB MNCs were harvested, and CPCs were identified utilizing the methods outlined in FIG. 4. In samples analyzed using the four-antibody panel and logarithmic dot plots (FIG. 4a-d), 0.290±0.218% (mean±s.d., n=10, range 0.170-0.900) of gated MNCs were CD31⁺CD34^{bright}CD45^{dim}AC133⁺. In comparison, when cell preparations from the same donors were analyzed using bi-exponential contour plots and FMO gating controls with the exclusion of monocytes, RBCs, and dead/apoptotic cells (FIG. 4e-i), CD31⁺CD34^{bright}CD45^{dim}AC133⁺ cells constituted only 0.134±0.0347% (mean±s.d., n=10, range 0.0800-0.200; logarithmic method vs. bi-exponential method, p=0.0380 by two-tailed, unpaired Student's t test) of viable glyA⁻CD14⁻ cells. FIG. 4g reveals a distinct CD31⁺CD34^{bright}CD45^{dim}AC133⁻ population that is indistinguishable from the neighboring CD31⁺CD34^{bright}CD45^{dim}AC133⁺ population in FIG. 4b. Exclusion of unwanted events, contour plots, bi-exponential scaling and an antigen panel chosen for maximal resolution of dull populations culminates in the enhanced resolution seen in FIG. 4g, facilitating the detection of unreported heterogeneity within the putative CPC population. FMO gating controls provide an objective means of distinguishing between a broad negative population and an adjacent dull positive subset and, in this case, confirm the visually apparent demarcation of AC133 expression. The combined frequencies of the CD31⁺CD34^{bright}CD45^{dim}AC133⁻ and CD31⁺CD34^{bright}CD45^{dim}AC133⁺ populations illustrated in FIG. 4g, confirm the observed frequency gated by traditional methodology in FIG. 4b, 0.310±0.024% (mean±s.d., n=10, range 0.270-0.400; logarithmic method vs. bi-exponential method, p=0.7973 by two-tailed, unpaired Student's t test). The putative CPC population enumerated by traditional flow cytometry methods includes two phenotypically distinct cellular subsets. Application of PFC analysis for CPC enumeration reveals novel heterogeneity and produces a smaller range of values with a lower standard deviation, which is critical for clinical comparative studies.

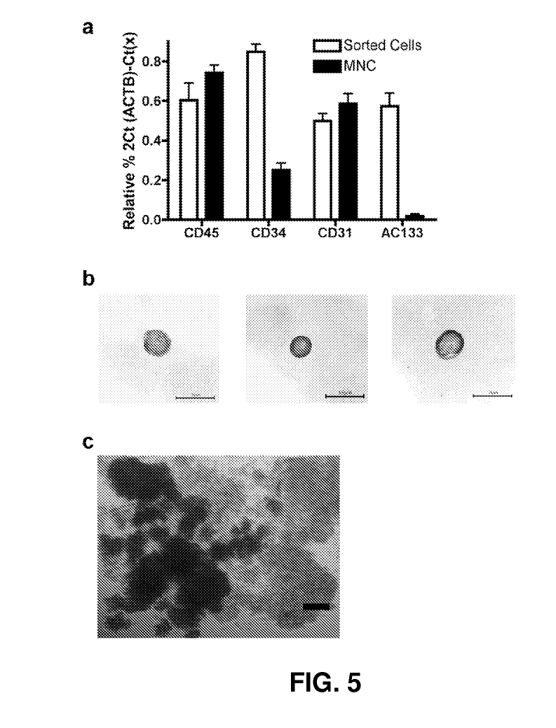

The functional identity of the progenitor cells that may correlate with extent of tumor progression and CVD risk is unknown. Experiments were designed and performed herein to determine the functional phenotype of CPCs in human PB identified with PFC. To better ascertain the identity of the CPCs, CPCs were isolated, pelleted, re-suspended, and deposited onto slides and Wright-Giemsa staining was performed on the CD31⁺CD34^{bright}CD45^{dim}AC133⁺ cells. Morphological analysis revealed hematopoietic blast cells or progenitor cells, which represent hematopoietic stem and progenitor cells (HSPCs) (FIG. 5b). Based on expression of multiple hematopoietic cell surface antigens (CD34, CD45 and AC133) and cellular morphology via immunocytochemistry, it was tested whether CPCs displayed functional properties of primitive HSPCs or EPCs in colony forming assays. Both CD31⁺CD34^{bright}CD45^{dim}AC133⁻ and CD31⁺CD34^{bright}CD45^{dim}AC133⁺ CPC populations formed primitive hematopoietic progenitor cell colonies including low proliferative potential-colony forming cells (LPP-CFCs), high proliferative potential-CFCs (HPP-CFCs), and colony forming unit-granulocyte erythroid macrophage megakaryocyte colonies (CFU-GEMMs, FIG. 5c) at a frequency range of 1:7-1:20 in each population. Neither population of CPCs yielded ECFCs in established in vitro clonal assays or formed capillary-like tubes with lumens in Matrigeff and neither population formed vessels in vivo.

Since both CPC populations formed primitive multi-lineage hematopoietic cell colonies and expressed HSPC antigens, it was tested whether these populations in fact contained NOD/SCID engrafting HSCs. NOD/SCID mice were sub-lethally irradiated and subsequently transplanted intravenously with purified human mobilized PB (mPB) CD34⁺ cells (positive control for HSPCs), CD31⁺CD34^{bright}CD45^{dim}AC133⁺ or CD31⁺CD34^{bright}CD45^{dim}AC133⁻ CPCs. Mice were sacrificed after 8-12 weeks, and human cell engraftment was measured in the mouse bone marrow (BM) by the presence of human CD45⁺ cells using species-specific monoclonal antibodies. Transplanted mouse BM was also analyzed for the presence of human CD19, CD33 and CD34 expressing cells; markers used to determine multi-lineage potential of engrafted human cells in NOD/SCID mice. Mice transplanted with either CD31⁺CD34^{bright}CD45^{dim}AC133⁺ or CD31⁺CD34^{bright}CD45^{dim}AC133⁻ CPCs demonstrated multi-lineage engraftment, which is an indication of transplantable NOD/SCID repopulating HSPCs (Table 1). Thus, the so-called human CPCs includes a heterogenous mixture of HSPCs and is described as circulating HSPCs (CHSPCs) rather than CPCs.

### Example 5

### CPC Heterogeneity Corresponds with Disparate Angiogenic Potential

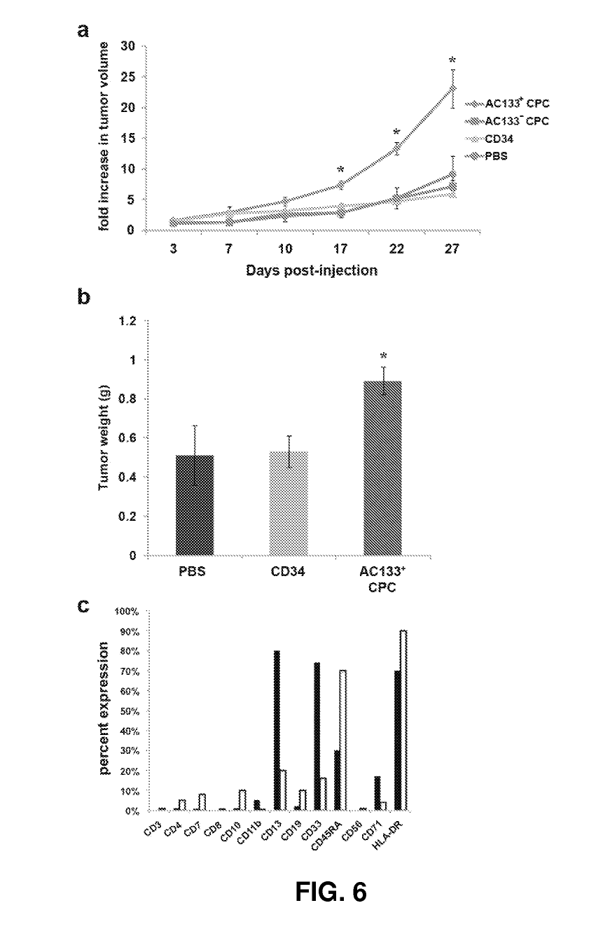

While the CD34⁺ cells, CD31⁺CD34^{bright}CD45^{dim}AC133⁺ CHSPCs or CD31⁺CD34^{bright}CD45^{dim}AC133⁻ CHSPCs all showed substantially similar rates of NOD/SCID engraftment, the capacity for promoting angiogenesis in an in vivo model was compared. Thus, NOD/SCID mice bearing human melanoma xenografts were intravenously injected with equal numbers of CB CD34⁺ cells, CD31⁺CD34^{bright}CD45^{dim}AC133⁺ CHSPCs or CD31⁺CD34^{bright}CD45^{dim}AC133⁻ CHSPCs and tumor growth was monitored over time in each cohort (FIG. 6a). Surprisingly, mice injected with CD31⁺CD34^{bright}CD45^{dim}AC133⁺ CHSPCs demonstrated a 23.12±0.15% (mean±sem n=8, range 18.35-29.00) fold increase in tumor growth as compared to tumor bearing animals treated with CD31⁺CD34^{bright}CD45^{dim}AC133⁻ CHSPCs (7.20±0.15% mean±sem n=8, range 5.31-9.87), the parental population of CD34⁺ cells (5.98±0.23% mean±sem n=8, range 4.99-7.59) and PBS control (9.17±0.14% mean±sem n=8, range 5.30-11.96; AC133⁺ CHSPCs vs. PBS, p=0.001 by two-tailed, unpaired Student's t test). When the explanted tumors were removed from the host mice in each cohort and weighed, animals injected with the CD31⁺CD34^{bright}CD45^{dim}AC133⁺ CHSPCs were significantly heavier (0.89±0.04 g mean±sem n=8) than the tumors removed from animals that were injected with PBS (0.51±0.06 g mean±sem n=8) or CD34⁺ cells (0.53±0.06 g mean±sem n=8; AC133⁺ CHSPCs vs. PBS or CD34⁺ cells, p<0.001) (FIG. 6b). Furthermore, the animals injected with the CD31⁺CD34^{bright}CD45^{dim}AC133⁺CHSPCs displayed a greater tumor vasculature that may resulted in the enhanced tumor volume measured in this group of animals. Though the transplanted CD31⁺CD34^{bright}CD45^{dim}AC133⁺ CHSPCs promoted tumor angiogenesis and tumor growth, there was no evidence of human CD31, CD33 or CD34 expressing cells/endothelium in the murine tumor vessels. Thus, CD31⁺CD34^{bright}CD45^{dim}AC133⁺ CHSPCs are pro-angiogenic and accelerate tumor growth in a statistically significant manner and are functionally distinct from the non-angiogenic CD31⁺CD34^{bright}CD45^{dim}AC133⁻ CHSPCs. Lineage phenotyping of pro-angiogenic and non-angiogenic CHSPCs revealed further heterogeneity (FIG. 6c). Pro-angiogenic CHSPCs expressed a preponderance of myeloid cell surface markers (CD11b, CD13, CD33) while the non-angiogenic CHSPCs displayed a preponderance of lymphoid cell surface markers (CD3, CD4, CD7, CD10, CD56) (FIG. 6c). This data demonstrates that both pro-angiogenic and non-angiogenic CHSPCs are not ECFCs or CPCs with endothelial potential but are distinct mixture of hematopoietic progenitor cells, myeloblasts, and HSCs with NOD/SCID repopulating ability.

### Example 6

### Ratio of Circulating Progenitor Subsets Denotes Disease State in Peripheral Vascular Disease and Breast Cancer Patients Treated with Sunitinib Malate

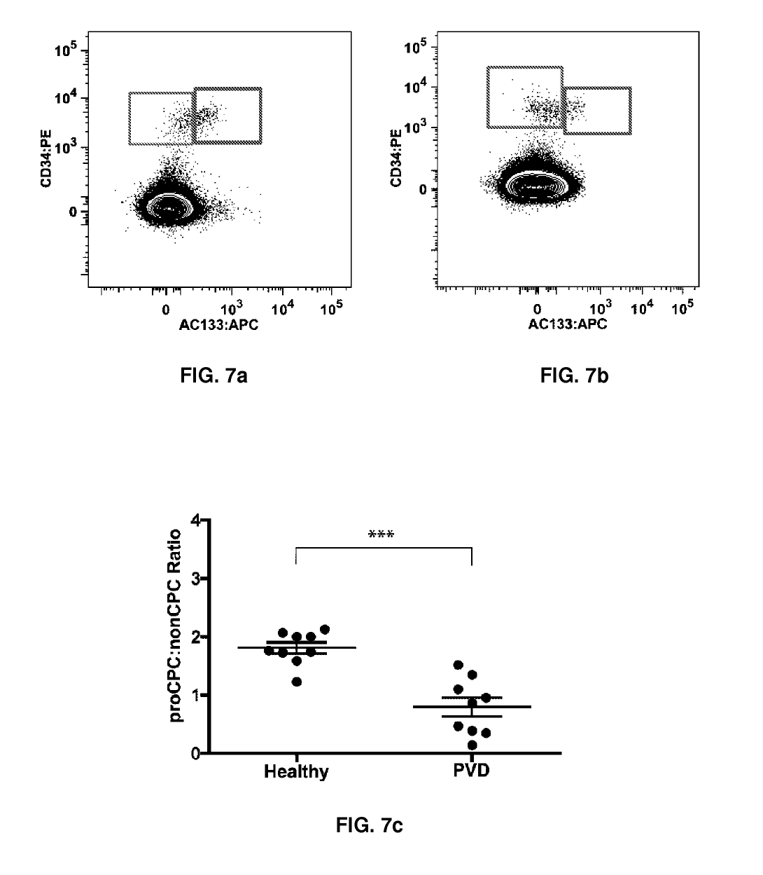

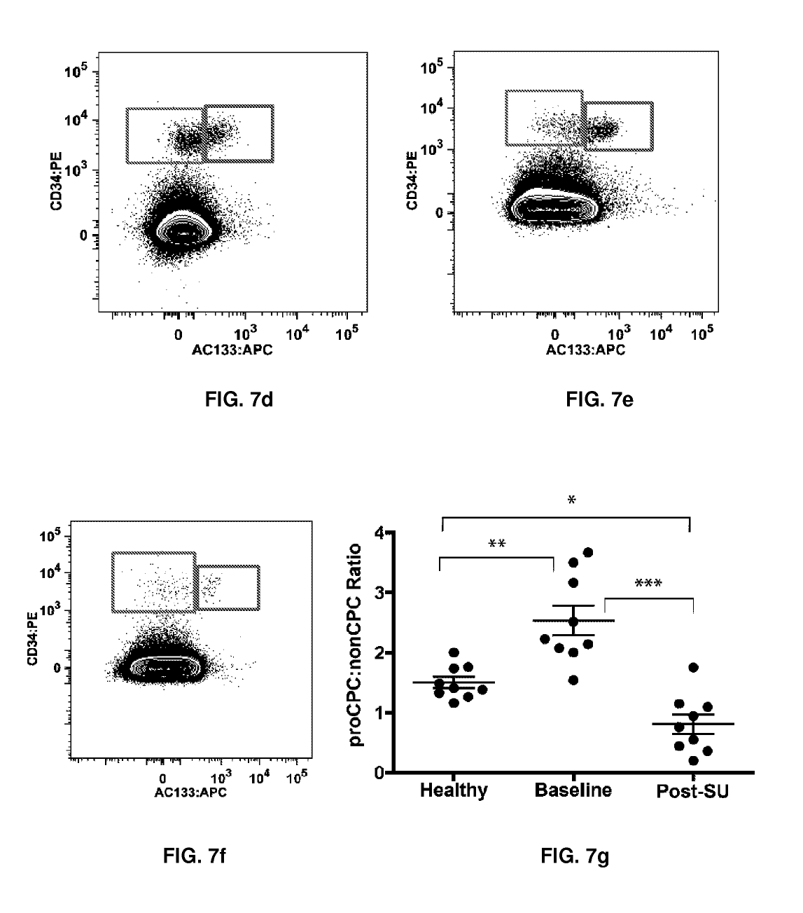

As disclosed herein, disparate angiogenic potential of the two CHSPC subsets (as functionally determined in the tumor xenograft model) was observed within the putative CPC population. It was tested whether these two progenitor fractions may regulate different aspects of vascular homeostasis. Observed disruptions in their relative frequencies may signal impaired or enhanced angiogenic function. The relative frequency of the pro-angiogenic CPC versus the non-angiogenic CPC was measured in two patient populations: peripheral vascular disease and metastatic breast cancer, representing different ends of the angiogenic spectrum. In patients with diagnosed peripheral vascular disease (PVD) a significant decrease in the ratio of pro-angiogenic CPCs to non-angiogenic CPCs (0.79±0.16050% mean±sem, n=9, range 0.140-1.52) was determined as compared to age and gender matched controls (1.81±0.09433% mean±sem, n=9, range 1.231-2.130; healthy vs. PVD, p=0.0001 by two-tailed, unpaired Student's t test)(FIG. 7a-c). In patients with actively growing breast tumors a significantly elevated ratio of the pro-angiogenic CPHSCs to the non-angiogenic CPCs (2.54±0.24559% mean±sem n=9, range 1.541-3.667) was observed as compared to a healthy controls (1.50±0.091257% mean±sem, n=9, range 1.161-2.000, p<0.01)(FIG. 7a-b,d). Additionally, assessment of these previously untreated patients with stage I, II or III histologically confirmed breast cancer following therapy with the small molecule receptor tyrosine kinase (RTK) inhibitor sunitinib malate reveals a significant decrease in the ratio of pro-angiogenic to non-angiogenic CPHSCs (0.807±0.16187% mean±sem, n=9, range 0.200-1.758, p<0.0001)—a shift that also correlates with a significant reduction in mean interstitial fluid pressure within the tumor (18.87 mmHg vs. 6.38 mmHg; p=0.002) (FIG. 7c-d). Patients with PVD or breast cancer and healthy controls are indistinguishable when the total progenitor population is compared. The wide population ranges of absolute cellular frequencies masks this distinct discrimination between patients and healthy controls. Therefore, the subsets that are truly biologically active in the process of tumor angiogenesis or in patients with vascular dysfunction are identified and analyzed herein.

### Example 7

### Cancer Therapeutics

As disclosed herein, in patients with actively growing breast tumors a significantly elevated ratio of the pro-angiogenic CPHSCs to the non-angiogenic CPHSCs (2.54±0.24559% mean±sem n=9, range 1.541-3.667) was observed as compared to a healthy controls (1.50±0.091257% mean±sem, n=9, range 1.161-2.000, p<0.01)(FIG. 7a-b,d). Agents to reduce the total number of pro-angiogenic CPHSCs are contemplated and thereby decreasing the ratio of the pro-angiogenic CPHSCs to the non-angiogenic CPHSCs is also contemplated.

### Example 8

### Cardiovascular Therapeutics

As disclosed herein, in patients with diagnosed peripheral vascular disease (PVD) a significant decrease in the ratio of pro-angiogenic CPHSCs to non-angiogenic CPHSCs (0.79±0.16050% mean±sem, n=9, range 0.140-1.52) was determined as compared to age and gender matched controls (1.81±0.09433% mean±sem, n=9, range 1.231-2.130; healthy vs. PVD, p=0.0001 by two-tailed, unpaired Student's t test)(FIG. 7a-c). Agents to selectively increase the total number of pro-angiogenic CPHSCs (e.g., at an ischemic site or a target site) are contemplated and thereby increasing the ratio of the pro-angiogenic CPHSCs to the non-angiogenic CPHSCs is also contemplated.

### Example 9

### Treatment Efficacy

As disclosed herein, in patients with actively growing breast tumors a significantly elevated ratio of the pro-angiogenic CPHSCs to the non-angiogenic CPHSCs (2.54±0.24559% mean±sem n=9, range 1.541-3.667) was observed as compared to a healthy controls (1.50±0.091257% mean±sem, n=9, range 1.161-2.000, p<0.01)(FIG. 7a-b,d). For patients undergoing cancer therapy, including chemotherapy, radiation or radiotherapy, and antibody therapy, treatment efficacy and prognosis are determined by monitoring the ratio of the pro-angiogenic CPHSCs to the non-angiogenic CPHSCs both during and after treatment.

### Materials and Methods

Blood Samples: PB samples (16-32 ml) were collected from 20 healthy adult donors (10 male and 10 female, age range 20-40 years) and CB samples (20-100 ml) were collected from 15 full-term newborns into citrate CPT Vacutainer tubes (BD Biosciences, Franklin Lakes, N.J., USA). The Institutional Review Board at the Indiana University School of Medicine approved all protocols, and informed consent was obtained from all donors. Granulocyte colony stimulating factor (G-CSF) mPB CD34⁺ cells were kindly provided through a Program of Excellence in Gene Therapy grant from Shelly Heimfeld at the Fred Hutchinson Cancer Research Centre, Seattle, Wash., USA.

Clinical Samples and Subject Characteristics: PB samples (16 mls) were collected from 9 patients (pts) with peripheral vascular disease (PVD) (6 male and 3 female, age range 50-81) along with age and gender matched controls. PVD pts ranged in Rutherford class 1-6, with co-morbidity CAD seen in 4 patients, 3 of which also displayed COPD. Patients with newly diagnosed stage Ic-IIIc breast cancer were treated with sunitinib monotherapy (100 mg Day 1; 37.5 mg D2-14) prior to the initiation of paclitaxel (80 mg/M² D 1, 8, 15 every 28 days×4 cycles) with sunitinib (25 mg/d). PB samples (48 ml) were collected from 9 patients (female, age range 39-59) at baseline, day 14 following sunitinib monotherapy, and at the end of cycle 6 along with age and gender matched controls. IFP was measured in three separate areas of the tumor using a micropres sure transducer catheter; mean and highest IFP recorded were analyzed.

Isolation of Mononuclear Cells: MNCs were isolated using the CPT Vacutainer system. Immediately following blood collection, CPT Vacutainer tubes were centrifuged at 1,600 g for 30 minutes at room temperature. The resulting hazy layer of MNCs, located just above the gel barrier within the tube, was removed and washed two times in phosphate buffered saline without calcium or magnesium (PBS, Invitrogen, Grand Island, N.Y., USA) with 2% fetal bovine serum (FBS, Hyclone, Logan, Utah, USA). Cells were counted on a hemacytometer.

Microvesicle Enrichment: PB collected in CPT Vacutainer tubes was centrifuged at 1,600 g for 30 minutes. The entire serum and MNC phase located above the gel barrier within the tube was removed and centrifuged at 13,000 g for 2 minutes. The resulting supernatant was transferred to a new tube and centrifuged at 18,000 g for 20 minutes to pellet microvesicles. The microvesicle pellet was resuspended in PBS with 2% FBS for antibody staining and flow cytometry analysis.

Antibodies and Staining Reagents: The following primary conjugated monoclonal antibodies were used: anti-human CD31 fluoroscein isothyocyanate (FITC, BD Pharmingen, San Diego, Calif., USA, cat. no. 555445), anti-human CD34 phycoerythrin (PE, BD Pharmingen, cat. no. 550761), anti-human AC133 allophycocyanin (APC, Miltenyi Biotec, Auburn, Calif., USA, cat. no. 130-090-826), anti-human CD14 PECy5.5 (Abcam, Cambridge, Mass., USA, cat. no. ab25395), anti-human CD45 APC-AlexaFluor (AF) 750 (Invitrogen, cat. no. MHCD4527), anti-human CD235a (glyA, R&D Systems, Minneapolis, Minn., USA, cat. no. MAB1228) conjugated to Pacific Blue (PacB, Invitrogen), anti-human CD3 FITC (BD Pharmingen, cat. no. 555339), anti-human CD4 FITC (BD Pharmingen, cat. no. 555346), anti-human CD7 FITC (BD Pharmingen, cat. no. 555360), anti-human CD8 FITC (BD Pharmingen, cat. no. 555634), anti-human CD10 FITC (BD Biosciences, cat. no. 340925), anti-human CD 11b PECy7 (BD Pharmingen, cat. no. 557743), anti-human CD13 (BD Pharmingen, cat. no. 558744), anti-human CD19 PE (BD Pharmingen, cat. no. 555413), anti-human CD33 APC (BD Pharmingen, cat. no. 551378), anti-human CD34 PECy7 (BD Biosciences, cat. no. 348791) anti-human CD41a APC (BD Pharmingen, cat. no. 559777), CD45RA FITC (BD Pharmingen, cat. no. 555488), CD56 CD71 HLA-DR FITC (BD Pharmingen, cat. no. 555811) IgG FITC (BD Pharmingen, cat. no. 555748), IgG PE (BD Pharmingen, cat. no. 555749), IgG APC (BD Pharmingen, cat. no. 555751), IgG PECy5.5 (Invitrogen, cat. no. MG118), IgG APC-AF750 (Invitrogen, cat. no. MG127), IgG PacB (Invitrogen, cat. no. S-11222), the amine reactive viability dye, ViViD (Invitrogen), and DAPI (Invitrogen).

In order to resolve the rare and/or dim populations of interest, specific antigen and fluorochrome conjugate coupling was optimized for the six-antibody plus viability marker staining panel described below.

PFC Immunostaining: A total of 10⁷ PB MNCs were suspended in 720 μl PBS with 2% FBS and incubated for 10 minutes at 4° C. with 180 μl human Fc blocking reagent (Miltenyi Biotec). Subsequently, 100 μl of the cell suspension was distributed into nine sample tubes with the following pre-titered antibodies: (1) unstained; (2) Isotypes: 4 μl IgG FITC, 15 μl IgG PE, 10 μl IgG APC, 10 μl IgG PECy5.5, 5 μl IgG APC-AF750 and 4 μl IgG PacB; (3) FITC FMO: 15 μl CD34 PE, 10 μl AC133 APC, 10 μl CD14 PECy5.5, 5 μl CD45 APC-AF750, 4 μl glyA PacB and 1 μl ViViD; (4) PE FMO: 4 μl CD31 FITC, 10 μl AC133 APC, 10 μl CD14 PECy5.5, 5 μl CD45 APC-AF750, 4 μl glyA PacB and 1 μl ViViD; (5) APC FMO: 4 μl CD31 FITC, 15 μl CD34 PE, 10 μl CD14 PECy5.5, 5 μl CD45 APC-AF750, 4 μl glyA PacB and 1 μl ViViD; (6) PECy5.5 FMO: 4 μl CD31 FITC, 15 μl CD34 PE, 10 μl AC133 APC, 5 μl CD45 APC-AF750, 4 μl glyA PacB and 1 μl ViViD; (7) APC-AF750 FMO: 4 μl CD31 FITC, 15 μl CD34 PE, 10 μl AC133 APC, 10 μl CD14 PECy5.5, 4 μl glyA PacB and 1 μl ViViD; (8) V450 Channel FMO: 4 μl CD31 FITC, 15 μl CD34 PE, 10 μl AC133 APC, 10 μl CD14 PECy5.5, and 5 μl CD45 APC-AF750; and (9) full panel: 4 μl CD31 FITC, 15 μl CD34 PE, 10 μl AC133 APC, 10 μl CD14 PECy5.5, 5 μl CD45 APC-AF750, 4 μl glyA PacB and 1 μl ViViD (six-antibody/viability marker panel). Cells were incubated with antibodies for 30 minutes at 4° C., washed twice in PBS with 2% FBS, and fixed in 300 μl 1% paraformaldehyde (Sigma Aldrich, St. Louis, Mo., USA). Additionally, anti-mouse Ig BD CompBeads (BD Biosciences, Bedford, Mass., USA) were stained with each of the individual test antibodies to serve as single-color compensation controls. Prior to use, each lot of antibody was individually titered to determine the optimal staining concentration. In some experiments, cells were incubated with either glyA PacB or ViViD to determine the individual contribution of RBCs or dead/apoptotic cells, respectively. Microvesicle enriched preparations of PB were stained with DAPI in combination with CD34 PE, CD45 APC-AF750, CD31 FITC, and/or CD41a APC for further characterization of microvesicles. All centrifugation steps for the immunostaining of microvesicle-enriched samples were performed at 18,000 g.

Flow Cytometry Acquisition and Sorting: Stained MNC samples were acquired on a BD LSRII flow cytometer (BD, Franklin Lakes, N.J., USA) equipped with a 405 nm violet laser, 488 nm blue laser and 633 nm red laser (for filter specifications see Supplemental Table 1). Prior to acquiring any data, photomultiplier tube (PMT) voltages were calibrated to the highest signal to background ratio based upon the antibody-fluorochrome pairs of interest. To ensure reproducibility, Sphero 1× rainbow bead controls (Spherotech) with established MFI were used daily to account for PMT voltage drift. At least 300,000 events were acquired for each sample. Data was acquired uncompensated, exported as FCS 3.0 files and analyzed using FlowJo software, version 8.7.3 (Tree Star, Inc., Ashland, Oreg., USA).

In some experiments, MNC samples stained with the six-antibody/viability marker panel were sorted on a BD FACSAria equipped with a 405 nm violet laser, 488 nm blue laser and 633 nm red laser (for filter specifications see Supplemental Table 1). BD CompBeads stained with the individual test antibodies were used as compensation controls. Automated compensation was applied using BD FACSDiva software, version 6.1.1. Populations of interest were sorted for purity into either a 15 ml conical tube or 24-well tissue culture plate.

Colony Assays: To assess the hematopoietic progenitor colony forming potential of CD31⁺CD34^{bright}CD45^{dim}CD133⁺ cells from PB, 500 freshly sorted cells or 10,000 MNCs were suspended in 0.66% to 1.0% agar (Becton Dickinson) in the presence of 1000 U/ml human interleukin (IL)-1α, 200 U/ml human IL-3, 100 ng/ml human macrophage colony stimulating factor (M-CSF), and 100 ng/ml human stem cell factor (SCF) (all from Peprotech, Rocky Hill, N.J., USA). Cells were plated in 35 mm Petri dishes in triplicate and scored for HPP- and LPP-CFCs on day 14.

Sorted sub-populations were also assayed for the presence of multi-potential granulocyte, erythroid, macrophage, megakaryocyte progenitors (i.e. CFU-GEMMs) using MethoCult® GF H4434, Complete Methylcellulose Kit (StemCell Technologies, Vancouver, BC, Canada) according to the manufacturer's protocol. PB CD31⁺CD34^{bright} CD45^{dim}CD133⁺ cells or MNCs were suspended in Iscove's Modified Dulbecco's medium (IMDM)+2% FBS, mixed with complete MethoCult® media, and plated into 35 mm Petri dishes in triplicate at 500 cells/plate for sorted cells or 10,000 cells/plate for MNCs. CFU-GEMMs were quantified by visual inspection on day 14.

To analyze the presence of ECFCs within FACS sub-populations, 5,000-10,000 CD31⁺CD34^{bright}CD45^{dim}CD133⁺ cells were suspended in complete EGM-2 (EBM-2 basal media, Lonza, Walkersville, Md., USA) supplemented with the entire EGM-2 growth factor bullet kit (Lonza), 10% FBS, and 1% penicillin/streptomycin (Invitrogen) and plated in one well of a collagen type I coated 96-well plate (BD Biosciences). Cells were cultured as described and examined daily by visual microscopy for ECFC colony growth until day 30.

Matrigel Tube Forming Assays: To assess the presence of functional endothelial cells within sub-populations of PB, freshly sorted CD31⁺CD34^{bright}CD45^{dim}CD133⁺ cells (3,500-5,000 per well) were seeded onto Matrigel-coated (BD Biosciences) 96-well plates. Wells were examined by visual microscopy every two hours for capillary-like tube formation. Early passage cultured ECFCs were used as a positive control.

Qpcr: For pre-amplification, 1,000 to 3,000 sorted cells were first lysed and reverse transcribed without direct RNA isolation exactly according to the manufacturer's protocol using the TaqMan PreAmp Cells-to-CT Kit (Applied Biosystems, Foster City, Calif., USA). All reverse transcription (RT) reactions were performed for 60 minutes at 37° C. and 5 minutes at 95° C. Pooled cDNA was pre-amplified with TaqMan Gene Expression Assays (Applied Biosystems) with primers for CD34, CD45, CD31, AC133 and ACTB (β-actin, Applied Biosystems). Pre-amplification was performed for 10 minutes at 95° C., 10 cycles of 15 seconds at 95° C. and 4 minutes at 60° C.

A quantitative real-time PCR (qPCR) analysis was performed with the ABI PRISM 7500 sequence detection system (Applied Biosystems). All PCR reactions were run in 96-well optical reaction plates (Applied Biosystems) with TaqMan Gene Expression Master Mix containing ROX passive reference dye for the targets of interest, the ACTB endogenous control labeled with FAM dye and a non-fluorescent quencher. Cycling conditions consisted of a 2 minute hold at 50° C. for uracilo-N-glycosylase degradation, 10 minute hold at 95° C. for enzyme activation, 40 cycles of 15 second denaturation at 95° C., 1 minute annealing and elongation at 60° C. Relative quantification of triplicate samples was performed using the delta-C_{T} method and expressed as fold increase relative to ACTB.

Cytospin Analysis: Cellular content of FACS CPC populations was evaluated by counting, cytospin preparation and Wright-Giemsa staining. Identification of cell types was done by visual inspection under 100× magnification and photomicrographs of cytospins were taken with an Olympus DP20 on an Olympus BX50 microscope.

Transmission Electron Microscopy: ViViD⁻CD14⁻glyA⁻CD3^{bright}CD34⁺CD45⁻AC133⁻ sorted sub-populations were allowed to settle on Nunc 10 mm, 0.4 μm polycarbonate membranes (Electron Microscopy Sciences, Hatfield, Pa., USA) and were fixed in 3% glutaraldehyde in cacodylate buffer (Sigma). After cutting out the filters, specimens were washed in cacodylate buffer and post-fixed in osmium tetroxide for 30 minutes. Specimens were rinsed in buffer, dehydrated in a graded alcohol series, and embedded in PolyBed 812 (Polysciences, Inc., Warrington, Pa., USA). Thin sections (80 nm) were cut with a diamond knife and stained with uranyl acetate and lead citrate. Specimens were viewed and photographed in a Philips CM100 transmission electron microscope (FEI Company, Hillsboro, Oreg., USA).

For immunoelectron microscopy analysis, PFC sorted cells were spun down and fixed in 4% paraformaldehyde in 0.1M phosphate buffer, dehydrated through a graded series of ethyl alcohols and embedded in Unicryl (Electron Microscopy Sciences). Thin sections (70-90 nm) were mounted on formvar/carbon coated nickel grids. After drying, the grids are then placed into a blocking buffer for 60 minutes, then without rinsing placed into primary antibody polyclonal anti-von Willebrand factor (vWF) (Abcam) diluted at 1:200 in 1% BSA/PBS overnight at 4° C. After rinsing with buffer the grids are then placed into the secondary antibody with attached 10 nm gold particles (AURION, Hatfield, Pa., USA) for 2 hours at room temperature. After rinsing in buffer the grids are placed in 2.5% glutaraldehyde in 0.1M phosphate buffer for 5 minutes, rinsed with buffer and distilled water, allowed to dry and stained for contrast using uranyl acetate. The grids are viewed with a Tecnai G 12 Bio Twin transmission electron microscope (FEI) and images taken with an AMT (Advanced Microscopy Techniques, Danvers, Mass., USA) CCD camera.

Immunomagnetic Isolation of CB CD146⁺CD45⁻ Cells: CB MNCs expressing the CD45 antigen were immunomagnetically selected using the human CD45 MicroBeads and Magnetic Cell Sorting (MACS) system (Miltenyi Biotec, Auburn, Calif., USA, cat. no. 130-045-801) exactly as directed by the manufacturer. The subsequent CD45⁻ cell sub-population was collected and a cell count and viability was assessed by trypan blue (Sigma Aldrich) staining. The CD45⁻ cells expressing CD146 were then immunomagnetically selected using the human CD146 MicroBead kit and MACS system (Miltenyi Biotec, cat. no. 130-093-596) exactly as directed by the manufacturer. The purity of MACS-separated sub-populations was confirmed by PFC acquisition and analysis.

To investigate the presence of ECFCs within MACS-sorted sub-populations, 50,000 CD45⁺ cells, CD146⁻CD45⁻ cells or CD146⁺CD45⁻ cells were plated into a 24-well collagen coated plate in cEGM-2 and cultured as described above. 30×10⁶ CB MNCs from the same donor were cultured in parallel as a positive control.

Mice: NOD/SCID mice, 6-8 weeks-old, were obtained from the Indiana University School of Medicine In Vivo Therapeutics Core and housed according to protocols approved by the Laboratory Animal Research Facility (LARC) of the Indiana University School of Medicine. All studies were conducted according to protocols approved by LARC and adhered strictly to National Institutes of Health guidelines for the use and care of experimental animals. All animals were fed doxycycline containing food pellets 1 week before transplantation and maintained on this feed for approximately 4 weeks after transplantation.

Transplantation of NOD/SCID Mice: All animals were given a sub-lethal dose of 300 cGy total body irradiation 4 hours before transplantation. The mPB CD34⁺ cells (10⁵ per mouse), sorted CD31⁺CD34^{bright}CD45^{dim}AC133⁺ sub-population (i.e. CPCs) from mPB CD34⁺ cells (10⁵ per mouse), or cells not contained in the CD31⁺CD34^{bright}CD45^{dim}AC133⁺ sort gate (i.e. non-CPCs) (2.5×10⁴ per mouse) were re-suspended in PBS and transplanted by tail vein injection. To assess engraftment, mouse BM cells were isolated from both femurs using aseptic procedures 8-12 weeks after transplantation. A total of 2×10⁶ cells were stained with anti-human CD45 APC-AF750 and anti-human CD34 PE antibodies, or anti-human CD45 APC-AF750, anti-human CD19 PE and anti-human CD33 APC antibodies. Approximately 500,000 events per sample were collected on a BD LSRII flow cytometer. Analysis was performed with FlowJo software version 8.7.3.

Determination of human CPC function in a melanoma xenograft model. NOD.CB17-Prkdc^{scid}/J (NOD/SCID) mice were subcutaneously injected with 2×10⁶ C32 human melanoma cells (ATCC) and tumor growth monitored. Once tumors reached ˜50 mm³, mice were injected with 5×10⁴ CPCs, nonCPCs, bulk CD34⁺ cells, or vehicle control (PBS). Tumor growth was monitored by caliper and the volume determined by the following formula: mm³=(width)²×length×0.5. The fold increase in tumor growth was determined by comparing tumor volume over time to the base line tumor volume. At the end of the experiment, mice were euthanized, tumors harvested, and the weight of each tumor determined. Data are presented as the mean+/−S.E. Statistical significance was determined using a 2-sided student's t-test to calculate p values.

Statistical Analysis: Statistical analysis was performed using GraphPad Prism software, version 5.01 for Windows (GraphPad Software, San Diego, Calif., USA). Data was tested for normality using the D'Agostino-Pearson normality test (alpha=0.05), and normal data sets were compared using two-tailed Student's t test or one-way ANOVA.

TABLE 1
Engraftment analysis of NOD/SCID mice transplanted with
mPB CD34⁺ cells, sorted CPCs or sorted non-CPCs.

| | | % of CD45⁺ | % of CD45⁺ | % of CD45⁺ |
|---|---|---|---|---|
| | % | cells that are | cells that are | cells that are |
| | CD45⁺ | CD34⁺ | CD33⁺ | CD19⁺ |
| CPC Mouse 1 | 6.00 | 10.7 | 12.9 | 14.4 |
| CPC Mouse 2 | 4.12 | 18.6 | 12.6 | 17.4 |
| CPC Mouse 3 | 4.78 | 9.22 | 13.2 | 14.2 |
| non-CPC Mouse 1 | 6.03 | 10.9 | 15.7 | 17.3 |
| non-CPC Mouse 2 | 6.85 | 12.0 | 15.3 | 16.7 |
| mPB CD34⁺ | 4.66 | 15.5 | 15.6 | 13.8 |
| Mouse 1 | | | | |
| mPB CD34⁺ | 4.35 | 15.2 | 13.2 | 14.1 |
| Mouse 2 | | | | |
| mPB CD34⁺ | 3.74 | 22.3 | 19.7 | 27.2 |
| Mouse 3 | | | | |
| mPB CD34⁺ | 3.83 | 11.3 | 16.1 | 18.5 |
| Mouse 4 | | | | |

TABLE 2
Filter specifications for PFC using a BD LSRII or FACSAria.

| | Detector | Fluorochrome | Dichroic Filter | Band Pass |
|---|---|---|---|---|
| Laser | Name | Detected | (Long Pass) | Filter |
| Blue 488 nm | A | PECy7 | 735 | 780/60 |
| | B | PECy5.5 | 685 | 695/40 |
| | C | PE Texas Red | 600 | 610/20 |
| | D | PE | 550 | 575/26 |
| | E | FITC | 505 | 530/30 |
| | F | SSC | | 488/10 |
| Red 633 nm | A | APC-AF750 | 735 | 780/60 |
| | B | APC | 600 | 670/14 |
| Violet 405 nm | A | Pacific Orange | 505 | 530/30 |
| | B | ViViD | | 450/50 |

1. A method of diagnosing cancer or peripheral vascular disease (PVD) in a subject, the method comprising determining the ratio of pro-angiogenic to non-angiogenic circulating hematopoietic stem and progenitor cells (CHSPC) and diagnosing that the subject has cancer if the ratio is higher or that the subject has PVD if the ratio is lower as compared to a reference value.

2. The method of claim 1, wherein the ratio of pro-angiogenic and non-angiogenic circulating hematopoietic stem and progenitor cells (CHSPC) is determined by polychromatic flow cytometry (PFC).

3. The method of claim 1, wherein the pro-angiogenic CHSPC are homogenously AC133⁺ and the non-angiogenic CHSPC are homogenously AC133⁻.

4. The method of claim 1, wherein the pro-angiogenic CHSPC are substantially homogenous for CD45^{dim}CD34⁺CD31⁺AC133⁺CD14⁻LIVE/DEAD⁻CD41a and the non-angiogenic CHSPC are substantially homogenous for CD45^{dim}CD34⁺CD31⁺AC133⁻CD14⁻LIVE/DEAD⁻CD41a⁻.

5. The method of claim 1, wherein the reference value is the ratio of pro-angiogenic to non-angiogenic CHSPC of a normal, healthy sample that is substantially free of cancer and PVD.

6. The method of claim 1, wherein the ratio of pro-angiogenic to non-angiogenic CHSPC is about 1.5 to about 3.6 for cancer and about 0.14 to about 1.52 for PVD.

7. The method of claim 1, wherein the pro-angiogenic CHSPC express a preponderance of myeloid markers selected from the group consisting of CD11b, CD13, and CD33 and the non-angiogenic CPCs express a preponderance of lymphoid markers selected from the group consisting of CD3, CD4, CD7, CD10, and CD56.

8. A method of diagnosing arterial disease, the method comprising identifying microvesicles that are substantially homogenous for CD31^{bright}CD34⁺CD45⁻AC133⁻ in a sample comprising mononuclear cells, wherein the microvesicles are not endothelial cells.

9. The method of claim 8, wherein the identification of microvesicles is by polychromatic flow cytometry (PFC).

10. The method of claim 8, wherein a substantial portion of the microvesicles is about 1-2 μm in diameter and are anuclear.

11. The method of claim 8, wherein the identified microvesicle population is substantially free of cells selected from the group consisting of myeloid progenitors, monocytes and macrophages.

12. The method of claim 8, wherein the arterial disease is cardiovascular disease.

13. The method of claim 8, wherein the microvesicles are selected from the group consisting of endothelial microvesicles that are DAPI⁻CD45⁻CD42b⁻CD31⁺LIVE/DEAD⁻, lymphoid microvesicles that are DAPI⁻CD45⁺CD42b⁻CD31⁻LIVE/DEAD⁻, and platelet microvesicles that are DAPI⁻CD45⁻CD42b⁺CD31⁻LIVE/DEAD⁻.

14. A method of enumerating circulating endothelial colony forming cells (ECFCs) in a blood sample, the method comprising identifying ECFCs that are homogenously CD34^{bright}CD45⁻ by polychromatic flow cytometry.

15. The method of claim 14, wherein the ECFCs are enumerated by bi-exponential scaling.

16. The method of claim 14, wherein the ECFCs form blood vessel in vivo through neoangiogenesis.

17. A method of reducing tumor growth or angiogenesis, the method comprising decreasing the number of pro-angiogenic circulating hematopoietic stem and progenitor cells (CHSPC) in a subject suffering from or suspected of having cancer.

18. The method of claim 17, wherein the cancer metastases is reduced.

19. The method of claim 17, wherein the pro-angiogenic circulating progenitor cells (CPC) is reduced by an anti-cancer agent.

20. The method of claim 17, wherein the anti-cancer agent is an angiogenesis inhibitor.

21. The method of claim 1, further comprising monitoring efficacy of anti-cancer treatment in a subject undergoing anti-cancer treatment.

22. The method claim 21, wherein the anti-cancer treatment is selected from the group consisting of chemotherapy, antibody therapy, and radiotherapy.
